(19) United States
(12) Patent Application Publication
(10) Pub. No.: US 2026/0324986 A1
(43) Pub. Date: Oct. 22, 2026

(54) DETECTING VASCULAR DISEASES IN SUBJECTS FROM ACOUSTIC MEASUREMENTS OF VEINS

(71) Applicant: University of Pittsburgh – Of the Commonwealth System of Higher Education, Pittsburgh, PA (US)

(72) Inventors: Timothy K. CHUNG, Pittsburgh, PA (US); Pete GUELDNER, Pittsburgh, PA (US); Cyrus J. DARVISH, Pittsburgh, PA (US); David A. VORP, Pittsburgh, PA (US)

(73) Assignee: University of Pittsburgh – Of the Commonwealth System of Higher Education, Pittsburgh, PA (US)

(21) Appl. No.: 19/121,389

(22) PCT Filed: Oct. 27, 2023
(86) PCT No.: PCT/US2023/036127

§ 371 Date: Apr. 15, 2025

Related U.S. Application Data
(60) Provisional application No. 63/420,344, filed on Oct. 28, 2022.

(51) Int. Cl. A61B 5/02; A61B 5/00; A61B 5/0205; A61B 5/11; A61B 7/02; B06B 1/06; G16H 50/20

(52) U.S. Cl. CPC A61B 5/02007; A61B 5/0053; A61B 5/0205; A61B 5/1118; A61B 5/6828; A61B 5/7257; A61B 5/7267; A61B 5/7275; A61B 7/026; B06B 1/0622; G16H 50/20; A61B 2562/04; B06B 2201/76

(57) ABSTRACT

Presented herein are related to systems and methods of determining likelihood of vascular conditions using acoustic measurements of veins in limbs of subjects. A pressure cuff may be secured about a region of a limb of a subject, and may apply pressure to the region to compress a vein in the limb. A plurality of sensors may be positioned on the region of the limb. Each sensor may acquire a respective acoustic signal in response to the pressure cuff releasing the pressure to the region. A computing system may receive the plurality of acoustic signals from the plurality of sensors. The computing system may generate, according to the plurality of acoustic signals, a plurality of metrics. The computing system may determine, according to the plurality of metrics, a likelihood of a vascular condition in the vein in the limb of the subject.

### CROSS REFERENCES TO RELATED APPLICATIONS

The present application claims benefit of priority to U.S. Provisional Application No. 63/420,344, titled “Artificial Intelligence Assisted Device to Diagnose the Severity of Deep Vein Thrombosis and Post Thrombotic Syndrome,” filed Oct. 28, 2022, which is incorporated by reference in its entirety.

### FIELD OF DISCLOSURE

The present disclosure is generally related to detecting vascular diseases in subjects using acoustic measurements of veins in limbs of such subjects.

### BACKGROUND

A computing device can use various models to analyze data to generate an output.

### SUMMARY

Aspects of the present disclosure are directed to systems and methods of determining likelihood of vascular conditions using acoustic measurements of veins in limbs of subjects. The system may include a pressure cuff secured about a region of a limb of a subject. The pressure cuff may apply pressure to the region to compress at least one vein in the limb of the subject. The system may include a plurality of sensors positioned on the region of the limb. Each sensor of the plurality of sensors may acquire, from the at least one vein, a respective acoustic signal of a plurality of acoustic signals in response to the pressure cuff releasing the pressure to the region. The system may include a computing system having one or more processors in communication with the plurality of sensors. The computing system may receive the plurality of acoustic signals from the plurality of sensors. The computing system may generate, according to the plurality of acoustic signals, a corresponding plurality of metrics. The computing system may determine, according to the plurality of metrics, a likelihood of a vascular condition in the at least one vein in the limb of the subject.

In some embodiments, the computing system may determine that the likelihood satisfies a threshold to identify the presence or absence of the vascular condition. In some embodiments, the computing system may provide, responsive the likelihood satisfying the threshold, an output to indicate the presence of the vascular condition of the at least one vein.

In some embodiments, the computing system may determine that the likelihood does not satisfy a threshold to identify the presence or absence of vascular condition. In some embodiments, the computing system may provide, responsive the likelihood not satisfying the threshold, an output to indicate the absence of the vascular condition in the at least one vein.

In some embodiments, the computing system may apply the plurality of metrics to a machine learning (ML) model, the ML model trained using a training dataset comprising a plurality of examples. Each example of the plurality of examples may identify (i) a respective second plurality of metrics generated from a second plurality of acoustic signals acquired from a respective limb, (ii) a label indicating a presence or absence of the vascular condition in the respective limb, and (iii) data derived from ultrasound imaging to measure a compliance of at least one respective vein in the respective limb.

In some embodiments, the computing system may receive clinical data associated with the subject, the clinical data identifying at least one of: (i) demographic information, (ii) usage of pharmaceutical, or (iii) co-morbidities. In some embodiments, the computing system may receive a clinical metric identifying one or more symptoms in the subject associated with the vascular condition. In some embodiments, the computing system may determine, in accordance with at least one of the clinical data or the clinical metric, the likelihood of the vascular condition in the at least one vein.

In some embodiments, the computing system may convert the plurality of acoustic signals from a time domain to a frequency domain to generate the plurality of metrics, each of the plurality of metrics identifying a respective coefficients from a discrete Fourier series. In some embodiments, the computing system may aggregate acoustic measurements from the plurality of sensors into the plurality of acoustic signals according to a respective position of each sensor of the plurality of sensors on the region of the limb.

In some embodiments, the computing system may generate, according to a second plurality of acoustic signals acquired subsequent to administration of a treatment after acquisition of the plurality of acoustic signals, a corresponding second plurality of metrics. In some embodiments, the computing system may determine, according to the second plurality of metrics, a second likelihood of the vascular condition in the at least one vein in the limb of the subject. In some embodiments, the computing system may determine, based at least on the likelihood and the second likelihood, a progress metric of the vascular condition in the subject.

In some embodiments, the system may include a wearable activity tracker fittable around the region of the limb. The wearable activity tracker may include the pressure cuff and the plurality of sensors in communication with the computing system to send an indication of a change in the vascular condition in the limb of the subject. In some embodiments, the pressure cuff may be secured against the region comprising at least one of a calf region or a thigh region of a leg of the subject, or an arm of the subject. The pressure cuff may radially compress the region.

In some embodiments, the plurality of sensors may include a plurality of piezoelectric sensors arranged radially around the region. The plurality of piezoelectric sensors may acquire a plurality of acoustic signals through one or more layers of tissue, muscles, arteries, or veins in the limb of the subject. In some embodiments, the vascular condition may include at least one of: (i) deep vein thrombosis (DVT), (ii) post thrombotic syndrome (PTS), (iii) chronic venous insufficiency, (iv) peripheral vascular disease, (v) limb ischemia, (vi) phlebitis, (vii) thromboangiitis obliterans, or (viii) lymphedema.

### BRIEF DESCRIPTION OF THE DRAWINGS

FIG. 1: Example of acoustic piezoelectric sensors along the longitudinal axis to record the intensity of sound. Based on the waveforms, the results can enable discernment of disease state (DVT vs. PTS).

FIG. 2: Input data from the Villalta survey/score, measured data from the hardware device, and clinical data (demographics, comorbidities, etc.).

FIG. 3A: Acute DVT is diagnosed after a patient experiences pain and swelling. Anticoagulants are prescribed and compression therapy is performed to reduce the thrombosis.

FIG. 3B: After DVT treatment is completed, symptoms can re-emerge 3-6 months later when the patient may be evaluated using the Villalta scale and imaging.

FIG. 3C: PTS is diagnosed after the re-emerging symptoms are tabulated. At this point, the patient may have significant reduction to their quality of life and be less responsive to treatment due to chronic venous insufficiency.

FIG. 4A: Healthy leg veins properly allow for one-directional flow upwards toward the heart. Stagnation of blood can lead to clots and thrombosis. Chronic venous insufficiency leads to vein shunt damage.

FIG. 4B: Schematic of proposed TDP sensor-based approach to detect acoustic signals from damaged veins.

FIG. 5A: Three piezoelectric sensor array embedded in PDMS silicon and attached a pressure cuff along with amplification boards that are connected to a microcontroller.

FIG. 5B: A thrombosis differentiating pressure (TDP) cuff. (Top Panel) PDMS-embedded pressure sensor arrays attached to the pressure cuff. (Bottom Panel) 3-D printed housing unit for the electronic components of TDP cuff including Arduino Nano boards, amplifiers, and power distribution block. A constant 5V power brick and USB hub is provided.

FIG. 5C: A diagram of a schematic of TDP sensor-based approach to detect acoustic signals from damaged veins

FIG. 5D: Signature waveform of a normal control patient (no DVT or PTS) when the cuff pressure was released after pressurization to 140 mmHg.

FIG. 5E: Raw waveform of a normal control patient (no DVT or PTS) when the cuff pressure was released after pressurization to 140 mmHg.

FIG. 5F: Processed waveform (e.g., smoothed) of a normal control patient (no DVT or PTS) when the cuff pressure was released after pressurization to 140 mmHg.

FIG. 6: The pressure cuff records various signals that are processed and tabulated along with the clinical data. Training data that is tabulated is used to train various multi-classification ML models to predict patient diagnosis (control, DVT, and PTS). The testing dataset is input into the trained models to test accuracy of classification.

FIG. 7 depicts a block diagram of a system for determining likelihood of vascular conditions using acoustic measurements of veins in limbs of subjects, in accordance with an illustrative embodiment.

FIG. 8A depicts a block diagram of a process to acquire acoustic measurements and derive metrics in the system for determining likelihood of vascular conditions, in accordance with an illustrative embodiment.

FIG. 8B depicts a block diagram of a process to determine vascular condition likelihoods in the system for determining likelihood of vascular conditions, in accordance with an illustrative embodiment.

FIG. 8C depicts a block diagram of a process to produce outputs in the system for determining likelihood of vascular conditions, in accordance with an illustrative embodiment.

FIG. 9 depicts a flow diagram of a method of determining likelihood of vascular conditions using acoustic measurements of veins in limbs of subjects, in accordance with an illustrative embodiment.

FIG. 10 depicts a block diagram of a server system and a client computer system in accordance with an illustrative embodiment.

### DETAILED DESCRIPTION

Following below are more detailed descriptions of various concepts related to, and embodiments of, systems and methods for determining likelihood of vascular conditions using acoustic measurements of veins in limbs of subjects. It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes.

Section A describes artificial intelligence assisted device to diagnose the severity of deep vein thrombosis and post thrombotic syndrome.

Section B describes machine learning model to predict and identify patients at risk of thrombosis.

Section C describes systems and methods for determining likelihood of vascular conditions using acoustic measurements of veins in limbs of subjects.

Section D describes a network environment and computing environment which may be useful for practicing various computing related embodiments described herein.

### A. Artificial Intelligence Assisted Device to Diagnose the Severity of Deep Vein Thrombosis and Post Thrombotic Syndrome

Deep vein thrombosis (DVT) is a condition where a blood clot forms in the deep veins of the body and occurs mostly in the legs. DVT affects 1 in 1000 adults annually and 10-30% of patients are to die within the first month of diagnosis. The clinical standard for diagnosing DVT relies on Duplex ultrasonography to detect blockages in the blood flow. Progress has been made to treat DVT; however, 1 in every 2-3 patients may develop post thrombotic syndrome (PTS) within 2 years of treatment. PTS is a condition that causes chronic pain and swelling of the leg. 5-10% of these patients may experience severe PTS, with some facing venous ulceration. PTS results in significant decreases in quality of life and has a major socioeconomic impact. Findings show that the immediate implementation of compressive socks can reduce the occurrence of PTS; however, there are currently no precise methods or guidelines for predicting when a patient may develop the chronic illness after treatment of DVT.

It is still unclear when a patient may develop PTS. The inflammatory response and recanalization after thrombosis can result in damages to the veins, resulting in venous reflux. The combination of reflux and vein obstruction can prevent adequate blood pumping back to the inferior vena cava and creating a hypertensive state observed in the veins. The high pressures result in tissue edema, fibrosis, and ulceration. In other approaches, a clinical process may include the monitoring of symptoms, imaging, and diagnosis via a quantitative scoring system, for example the Villalta score.

Compliance of the veins is critical for returning deoxygenated blood back to the heart. Both DVT and PTS damage vein, disallowing for optimal blood flow that is debilitating and potentially fatal for patients. Biomechanical stress analysis of DVT and PTS can potentially relay the severity and can provide a distinct biomarker to discern this critical transition (e.g., DVT to PTS). Utilizing knowledge and expertise, a system is developed to non-invasively measure the biomechanical behavior of diseased veins. The system may include a piezoelectric vibration/sound detection device, and may incorporate a machine-learning based solution to classify severity of disease state (normal, DVT, and PTS) with the following set of specific aims:

Identifying the local vein wall changes may lead to an understanding of the development of PTS. To address the limitations of predicting and preventing PTS, research is initiated to understand the mechanical changes in the vein wall. A device is disclosed herein, configured to non-invasively measure mechanical changes in the vein wall after DVT. The device may significantly improve the ability to predict the chronic illness and can ensure that patients can receive preventative treatment, improving the quality of life and reducing healthcare costs for people affected by DVT.

Ultrasound images can be segmented and reconstructed from patients who have recently been diagnosed with DVT and diagnosed with PTS. Of the patients recently diagnosed with DVT, there can be a group of those who have developed PTS and those who have not developed PTS. Finite element analysis can be conducted on all groups to gather stresses and quantify compliance by determining how much diameter changes with pressure. Compliance can be a determining mechanical factor for how damaged the vein is. Finally, whether there are any differences between groups may be determined.

Ultrasound can be used to assess compliance of the vein for three patient groups (e.g., healthy, DVT, and PTS). Vein segmentations from US is used to construct stress models using finite element analysis to quantify the wall stresses of the vein. Statistical analysis can be performed to compare potential differences between all groups (e.g., using compliance and wall stresses as the response variables). This dataset can later be used to validate the hardware device developed in Aim 2.

In addition, a pressure cuff device is developed utilizing an array of audio sensors to determine the compliance of the vein, which can be based on audio feedback and/or time-of-flight signal changes. The cuff can apply pressure while the sensors can produce a signal based on the sound of the blood flow.

A multi-positional array of acoustic piezoelectric sensors can be embedded into a pressure cuff along the longitudinal axis. The pressure cuff can pneumatically pressurize the outside of a calf to compress the vein, and subsequently released (e.g., for the veins to refill). The acoustic data can be recorded as the veins refill. The rate in which the sensor receives no acoustic signal may be directly correlated to the compliance of the vessel. For example, the refilling of a normal vein can be slower than in a diseased vessel as it is stiffer and does not allow for appropriate expansion due to low compliance. The position of the acoustic sensors can leverage latency between sensors to recover additional information about blood flow to detect regurgitation (e.g., an indication of PTS or poor valvular functionality). For example, since a healthy vein is to carry blood toward the heart, the regurgitation of blood flow in an unhealthy vein may correspond to a direction of blood flow away from the heart, as opposed to the expected direction of toward the heart.

After gathering mechanical and clinical data, patient specific markers can be provided as input to a machine learning model to predict the development of PTS. These can include patient data, a D-dimer test result, compliance measures, and/or audio signal feedback from the pressure cuff.

A machine learning multi-classification model can be trained using: (i) Pressure cuff data, (ii) Survey (e.g., using the Villalta score and individual categories), and (iii) Patient clinical data (e.g., demographics, BMI, co-morbidities, and blood test results that includes the D-dimer test). Once the model is trained, the model can be used to determine whether a patient has DVT, PTS, or is healthy (e.g., no DVT). Depending on the implementation and data collected in Aim 2, the developed hardware device can discern disease status. The classification model would not be needed if the pressure cuff could be independently validated to demonstrate accurate

### Diagnoses.

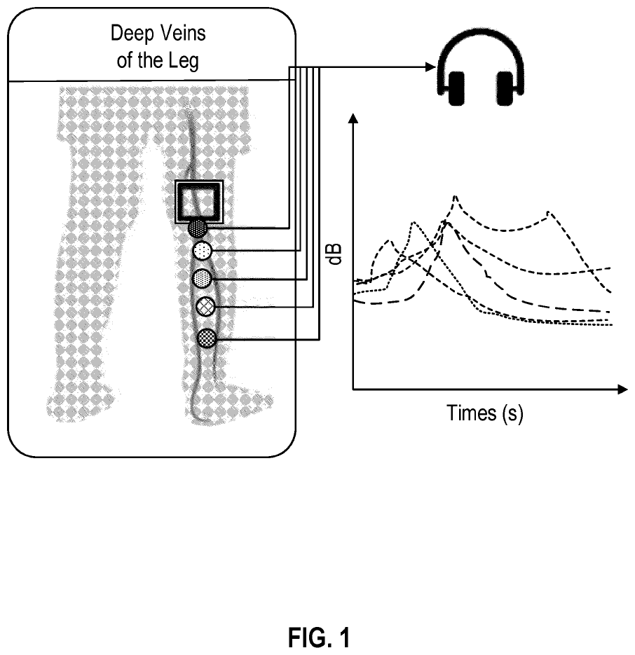

A hardware device that includes a pneumatically pressurized cuff to go around a portion of a limb, such as the calf or thigh of a patient. The pressure cuff includes a multi-positional array of piezoelectric sensors to detect vibrations or audio signals at various levels/bands/regions (e.g., around the circumference of the device and various axial positions, FIG. 1). The pressure cuff also records the local pressures required to compress the limb and vein for additional information for biomechanical analysis.

The signal received can be able to at least one of:
1) Detect local compliance changes in diseased veins and/or their respective severity (e.g., normal, DVT, and PTS) through audio signals.
2) Latency detection of peak sound or vibration intensity can allow for the detection of reflux across a diseased valve in the vein.
3) Calculate a hypertensive venous state determined by the sensors and pressure cuff during movement/walking (a feature that has been unavailable and a challenge to measure, based on clinical experts' feedback).

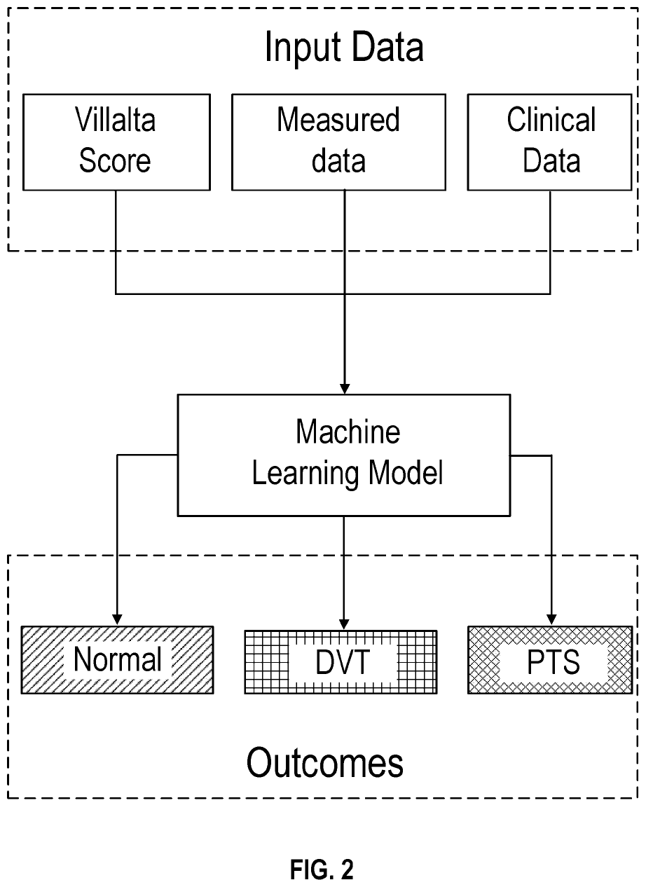

The hardware device can record data to record the biomechanical response of the veins. The data can be analyzed independently to provide disease status using the metrics recovered from a validation dataset. A secondary device feature allows for a software as medical device approach to be coupled with the primary hardware device using machine learning. A classification machine learning model can be used to striate patient diagnoses based on ‘normal’, ‘DVT’, and ‘PTS’ status' (e.g., illustrated in FIG. 2). Data from the hardware device can be processed/simplified to report single metrics (e.g., summarized waveforms using Fourier series or polynomial curve fitting approaches). In some embodiments, clinical data inputs can incorporate patient demographics, pharmaceutical usage, and co-morbidities (e.g., hypertension, other cardiac problems, etc.).

Machine learning multi-classification models can be trained iteratively to striate patient outcomes. Feature importance can be tabulated and can be used to reduce the number of inputs required for an accurate prediction. The ML approach may be unneeded depending on the sensitivity and specificity of the signal analysis from the hardware device.

Validation of the hardware and software approach. The hardware device relies on the ability of the pressure cuff to compress and/or depressurize the vein (e.g., that enables the acoustics signals to be produced). A database of ultrasound measurements/results can be used to interpret the corresponding signals from the veins. The database can also contain the diagnoses of DVT or PTS for additional validation of the corresponding signals.

### B. Machine Learning Model to Predict and Identify Patients at Risk of Thrombosis

Each year, 1 in 1000 of the general population are diagnosed with deep vein thrombosis (DVT), which can lead to life threatening pulmonary embolism, the third most common cause of cardiovascular death (approximately 300,000 each year). Diagnosing DVT can be achieved using medical imaging techniques via compression ultrasound or computed tomography (CT). Once diagnosed, patients with DVT can be treated using compression therapy and/or anti-coagulants. However, about 50% of symptomatic patients with DVT lesions may progress to post thrombotic syndrome (PTS), which is associated with chronic inflammation, pain, and venous ulcers. Patients that suffer with PTS have a reduced quality of life compared to those with DVT, due to the persistent pain in the lower limbs. Although DVT and PTS affect the general population, there is higher prevalence in the African American community.

There are significant costs associated with managing patients with PTS and even more relating to its downstream complications. For example, the average cost in the US for a venous ulcer in 1999 was roughly $10,000 per patient/year. While diagnosis and treatment for DVT is well established, there are no current methods to diagnose PTS or accurately predict or detect the transition from DVT to PTS. Such tools could enable clinicians to prevent PTS through aggressive patient management including increasing dosages of blood thinners. The present disclosure describes embodiments of a hardware and software device that can pre-emptively identify DVT patients at high risk of developing PTS. The device may be coupled with a machine learning (ML) model that can be used to predict patient outcomes for those affected by DVT, for instance by reducing progression to PTS through earlier diagnosis and treatment. Described herein are: (1) Measures of compliance and/or acoustic signals from a signature waveform along the longitudinal axis of a vein that are distinguishable between healthy, DVT, and PTS patients; and (2) A trained ML model that can reliably diagnose existing PTS and/or predict which DVT patients may transition to PTS. These may be tested/developed, respectively, through the following approaches:

Presented herein is a configuration of a non-invasive thrombosis differentiating pressure (TDP) cuff to measure compliance via axially varying acoustic signals from limb/leg veins and compare between healthy, DVT, and PTS patient groups. A pneumatically driven TDP cuff hardware device can be configured to be placed on the thigh or calf of a patient for instance, with confirmed DVT or PTS (or control patients with neither). The cuff may consist of a multi-positional array of piezoelectric sensors to detect acoustic signals along the axis of the underlying vein. The pressure cuff may include a strain gauge to detect deformation during pressurization and/or a pressure transducer to measure the applied cuff pressure to relay physical parameters to measured acoustic signals. Pressure inflation of the cuff may compress/constrict the vein wall and duplex ultrasound may be used to measure the diameter of the vein in the compressed state at each sensor position. Compliance measured from ultrasound may be used to correlate to the acoustic signals from the TDP cuff. These measurements may be quantitatively compared between positions, and among different patient groups: such as those confirmed to have i) control (no DVT or PTS), ii) DVT, or iii) PTS to identify each patient group's signature acoustic waveform.

Presented herein is a configuration of a machine learning (ML) model to predict and identify patients at risk of transition to PTS. A multi-classification ML model may be trained using inputs (independent variables) of compliance, vibration/audio waveform metrics, patient survey data (e.g., Villalta score), and/or selected patient clinical data (demographics, history, pharmaceutical-use, and co-morbidities). The various signals collected from the piezoelectric sensors may be post-processed and quantified using Fourier series and latency of peak signals. Training and testing may be performed using various python libraries that include sci-kit learn, XGBoost, TensorFlow, and algorithms in MATLAB (MathWorks, Natick, MA USA). Dimensionality reduction techniques may be used to reduce the total number of variables required to make an accurate prediction using principal component analysis and feature importance mapping. The output (e.g., dependent variable) may be a score to predict a patient's risk of developing PTS after an acute DVT episode. The trained model may be tested using data collected from the hardware device from patients that have been diagnosed with DVT or PTS to test accuracy, including the control (e.g., no DVT or PTS), DVT, or PTS patient groups.

In some embodiments, the disclosed solution combines methods of vascular biomechanics, electromechanical systems, and ML to develop or configure a novel, non-invasive hardware device that is paired with an innovative ML-based software system to predict whether DVT may transition to PTS. Accurate predictions would allow the adoption of a preemptive, more aggressive treatment plan to prevent this transition from occurring, ultimately reducing health care costs and improving patient outcomes (e.g., prevention of PTS would drastically improve the quality of life of DVT patients). This low-cost and non-invasive medical device can potentially reduce long-term healthcare costs, reduce incidence of PTS in minority communities, and/or reduce healthcare disparities.

Deep vein thrombosis (DVT) is a common cardiovascular disorder where a blood clot forms in the deep veins, typically in the lower leg or thigh. DVT afflicts as high as 3 in 1000 individuals in the general population each year and can lead to a pulmonary embolism (PE), the third most common cause of death of cardiovascular etiology. According to the CDC, at any given time there are 900,000 people affected with DVT in the United States and predominantly affects the Black community that suffer from disparities within healthcare. Approximately 300,000 patients die each year from DVT/PE. Clinical diagnosis of DVT can be accomplished using compression duplex ultrasound and in some rarer cases MRI or CT imaging. Approximately 50% of symptomatic DVT patients will progress to post thrombotic syndrome (PTS), a lingering disorder that is associated with chronic venous insufficiency. Common symptoms of PTS include chronic inflammation, pain, and venous ulcers. PTS also bears a significant socioeconomic impact, increasing cost to the patient when compared to DVT treatment alone=.

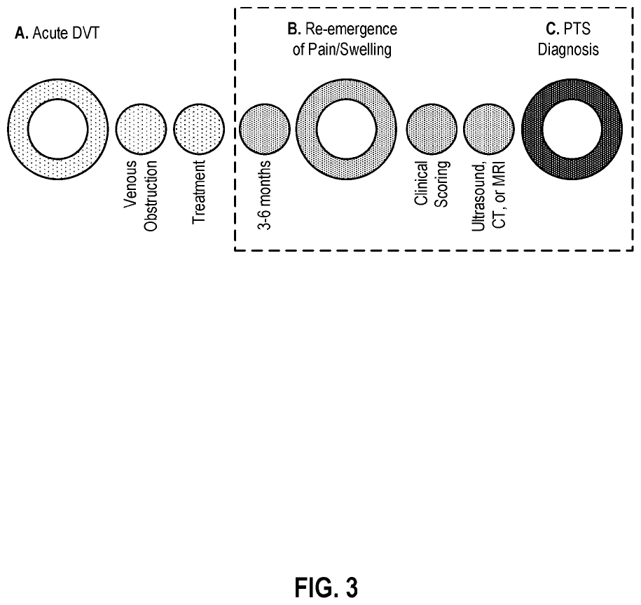

A typical patient pathway after the initial diagnosis of acute DVT to PTS is depicted in FIG. 3. PTS is diagnosed 3-6 months after the incidence of acute DVT and can only be diagnosed if the symptoms of DVT had previously subsided with treatment. Vein walls and valves experience damage after an episode of DVT which leads to the swelling and pain that is then diagnosed as PTS. Diagnosis of PTS is currently limited to an assessment of clinical symptoms using several clinical surveys (e.g., Widmer, Villalta, and Ginsberg). The most utilized diagnostic metric is the Villalta scale where clinical symptoms and signs that directly relate to the severity of PTS are tabulated. Items scored in the Villalta scale include symptoms such as pain, cramps, heaviness, and clinical signs such as pretibial edema, skin induration and venous ectasia. A patient is considered to have PTS if they score greater than five points on the scale, with greater than fifteen points considered as severe (10-15% of patients). The scoring system is accompanied with imaging, typically duplex ultrasound or in some cases CT venography or MRI with contrast to confirm chronic venous insufficiency. Air plethysmography is a separate method which explores volume changes with pressure to measure venous reflux and, potentially, DVT and PTS. However, this method has not been widely adopted in a clinical setting and cannot discern changes that lead to PTS, limiting the clinical utility of this approach.

There are several studies investigating the biomechanics of veins after a thrombotic event. Thrombotic damage leads to a thickening of the vein wall which in turn lowers the compliance of the vessel, impeding venous function. Therefore, compliance—a biomechanical measure to assess the ability of a vessel to distend under a change in pressure—can be used as a biomarker for venous functionality. This serves as the context for the disclosed solution; specifically, measures of compliance of affected veins can distinguish between healthy, DVT and PTS patients, which can subsequently/optionally be used to inform a predictive model for the transitioning of an acute DVT to PTS.

Analog to digital convertors (ADC) can be used in medical device applications to measure unique responses from a variety of analog sensors. Analog sensors can be used in medical devices to record data rapidly for monitoring and detection by converting the electrical signals to meaningful measurements e.g., voltage or current). The sensors that may be used in some embodiments of the disclosed hardware device can include piezoelectric sensors, strain gauge, and/or pressure transducers that are arranged in such a way as to non-invasively measure compliance. Piezoelectric sensors can convert mechanical stimulation into an electrical signal that can be converted into meaningful units. Piezoelectric sensors may be used to measure vibration or sound waves from the pressurization and depressurization of the proposed hardware device. Strain gauges can be mechanically deformed through a tensile or compressive load, where the overall resistance change directly corresponds to the voltage that is received by the ADC. A strain gauge may be used to measure the displacements that are introduced by the pressurization of the proposed hardware device. A pressure transducer may be used to measure to monitor the pressure (mmHg) during pressurization and depressurization of the hardware device. The pressure transducer may change its voltage as pressure increases. An array of piezoelectric sensors that synchronously measure pressure and strain changes in real-time may enable the hardware device to measure relative changes in compliance.

Artificial Intelligence (AI) is a broadly defined field that leverages different data types to provide solutions to perform tasks typically assigned to humans. Machine learning is a sub-category of AI that incorporates large quantities of pre-processed data to identify or predict an outcome. Classification algorithms seek to discriminate outcomes based on datasets, whereas regression algorithms output values of a specified variable. Machine Learning (ML) has continued to mature and become more robust in predicting desired outcomes using supervised or unsupervised learning. Ensemble gradient-boosted trees that use mixed data types can improve model performance. Data-driven ML models have been expanded for many different use cases and introduced in various biomechanical applications in vascular diseases. The use of ML for this project provides an approach to predict the likelihood of DVT patients transitioning to PTS.

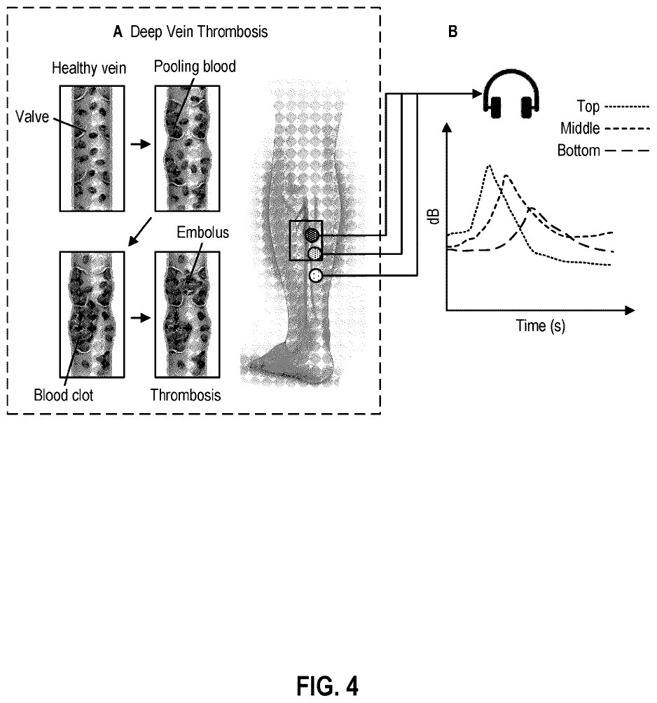

A significant gap remains as the transition from DVT to PTS is poorly understood. There are no imaging techniques available that would indicate that a patient would develop PTS given the current clinical progression of the disease. Disclosed herein is an innovative solution through the design and development of a low-cost Thrombosis Differentiating Pressure (TDP) cuff that can be coupled with a ML model to aid clinicians to predict patients that are likely to transition from DVT to PTS (FIG. 4). This approach can use a unique combination of hardware and software that can potentially halt or slow the transition from DVT to PTS. The hardware approach can use an array of analog sensors to extract a signature acoustic waveform from normal, DVT, and PTS patients. The software approach can utilize machine learning to detect minute differences in the signature waveforms and can incorporate/consider the overall health of each patient to predict their likelihood of developing PTS from DVT. This innovative solution can provide clinicians the opportunity to pre-emptively treat high-risk patients to halt or eliminate the progression to PTS. The non-invasive TDP cuff and ML model can serve as a predictive hardware device to better manage patients with DVT by changing the treatment regimen to reduce patients at risk of PTS, improve quality of life, reduce healthcare costs, and/or improve long-term patient outcomes.

Disclosed herein is a hardware and algorithmic approach to rigorously assess signature waveforms that are received by the disclosed hardware device. Presented herein include iterative methods to train various machine learning models that allows for the truncation or omission of certain variables by weighting their importance. Additional scrutiny to the models can be performed by separating potential factors such as sex, race, and certain co

There are no prior methods that allow for the prediction of DVT to PTS during an office visit, and duplex ultrasound can only confirm that venous insufficiency is chronic after the transition from DVT to PTS. It is believed that measurement and utilization in an ML-based model of affected vein wall compliance would give the ability to pre-emptively identify high-risk DVT patients (e.g., those that are likely to develop PTS). This would allow clinicians to alter treatment plans they would otherwise prescribe and could reduce the number of patients that transition from DVT to PTS. Although a non-invasive technique exists that assess volume changes (air plethysmography) to measure venous reflux, this methodology has not been widely adopted due to the standard use of duplex ultrasound. Further, measurement of volume changes alone is unable to distinguish patients with PTS. This method to correlate signature acoustic waveforms from veins and relate them to compliance or vein damage may allow for the extraction of a key biomechanics-based biomarker towards the development of a predictive model to identify patients that are at risk of developing PTS.

The TDP cuff may be pneumatically driven to radially compress a portion of a limb (e.g., the calf or thigh) and may include a multi-positional array of piezoelectric sensors to detect acoustic signals along the longitudinal axis of an underlying vein. The TDP cuff can include a pressure sensor and strain gauge to measure the change in the circumference with respect to pressure. The pressure inflation of the cuff may compress/constrict the vein wall, and duplex ultrasound may simultaneously measure the compliance of the veins. Compliance calculated from these measures as well as the signature acoustic waveforms, may be quantitatively compared between positions and among different patient groups: such as control (e.g., healthy, no DVT or PTS), DVT, and PTS. The duplex ultrasound measured compliance may be compared to the signal responses recorded from the TDP cuff. The comparison between ultrasound-measured compliance and the TDP cuff may provide the basis for converting the analog sensor data into a compliance biomarker. The results may be compared using various statistical approaches that include multiple regression analysis and three-way ANOVA.

The TDP cuff can include a pneumatically driven pressure cuff that can accommodate a wide range of calf and thigh diameters. A pressure cuff may be repurposed to integrate an electronically controlled pneumatic pump with a linked pressure transducer to provide continuous monitoring of pressure. Piezoelectric sensors may be placed radially around a patient's calf or thigh for instance, with multiple longitudinal positions (e.g., the craniocaudal axis when in the supine position). The piezoelectric sensors may be embedded into Polydimethylsiloxane (PDMS) using a custom 3D printed, or laser cut mold and connected to a custom printed circuit board (PCB) that is manufactured using a Voltera (Voltera Inc., Kitchener, Ontario, Canada). The array of piezoelectric sensors may be placed on the lateral and posterior portion of the calf/thigh region to record acoustic signals during the pressurization and depressurization of the TDP cuff. Additionally, strain gauges may be molded directly into the PDMS to measure local displacement/strain changes during pressurization and depressurization. Continuous analog sensor data may be acquired using an Arduino (Arduino, Turin, Italy) microcontroller and recorded for signal post-processing.

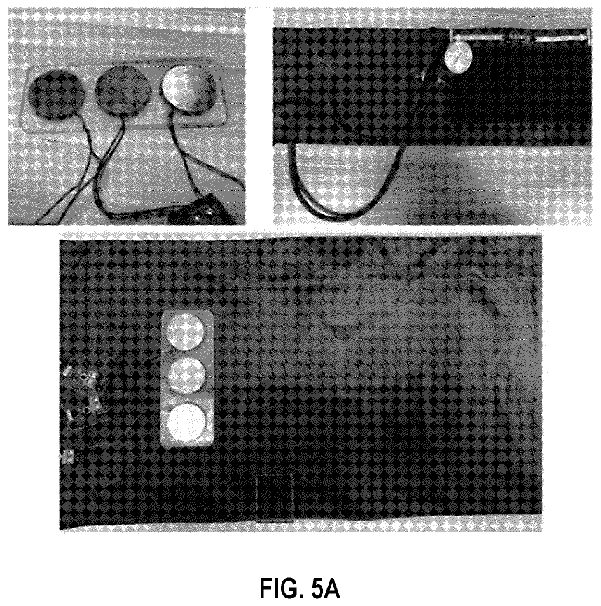

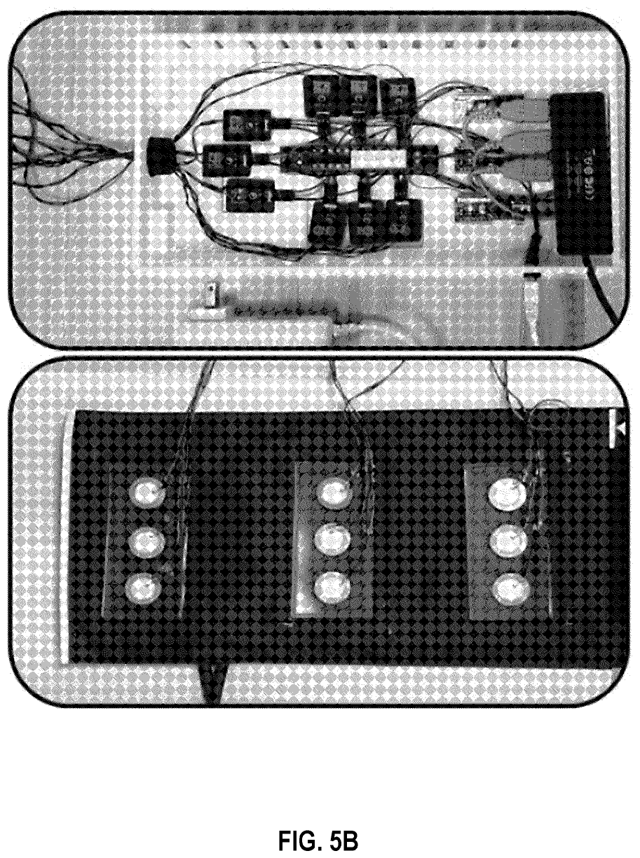

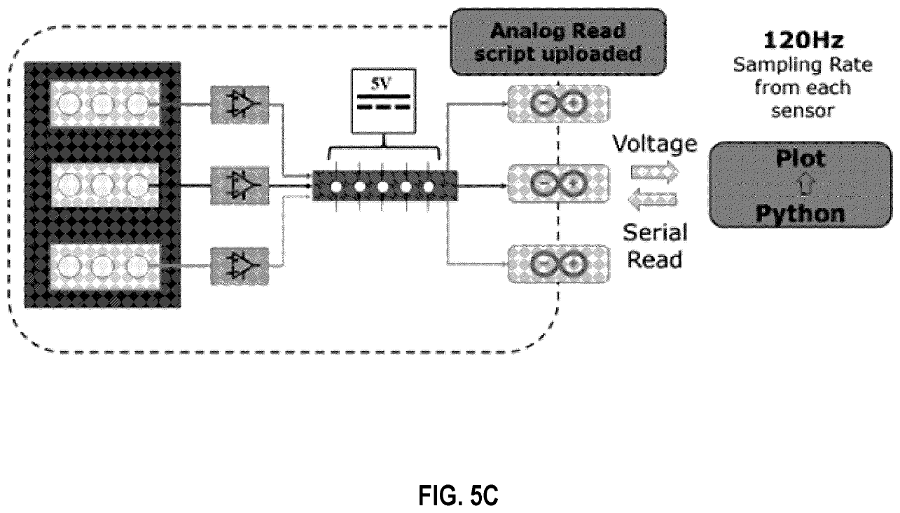

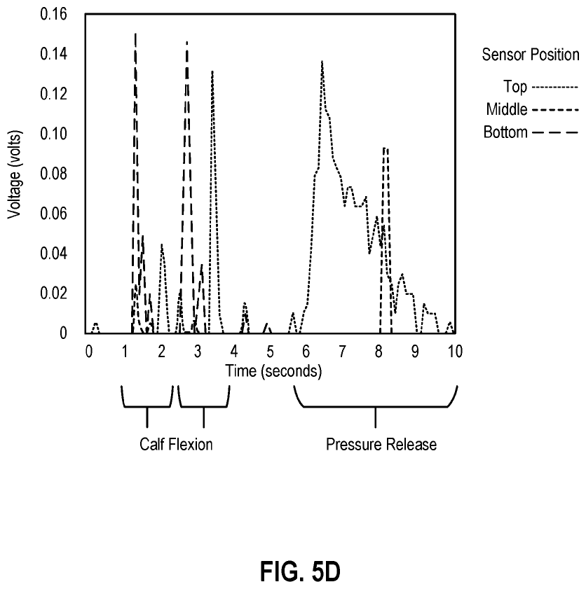

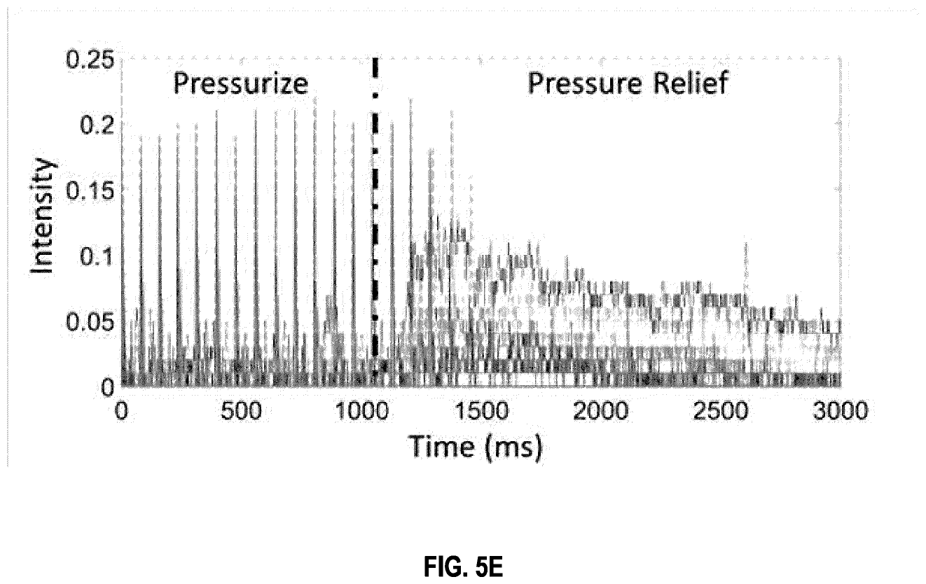

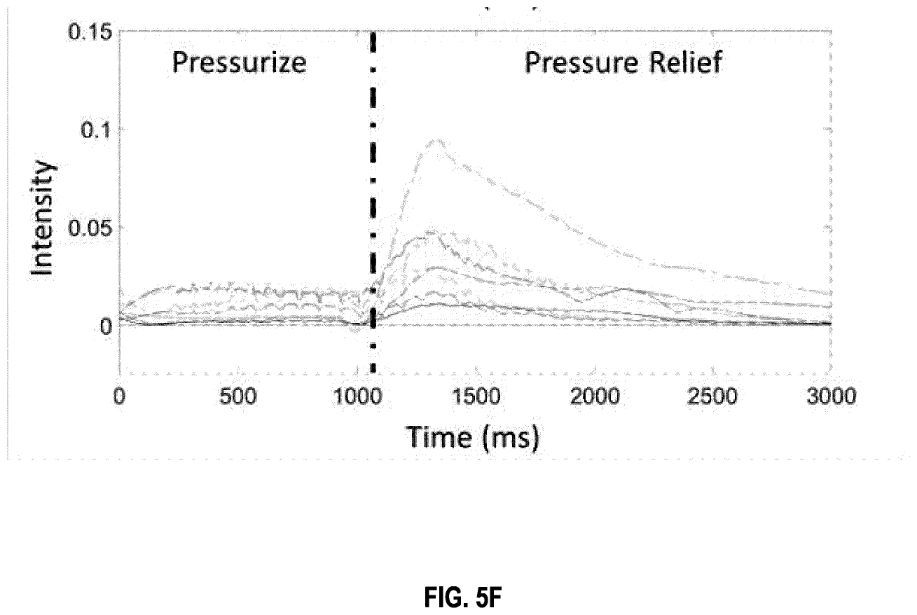

The approach can include a pressure cuff and can mold/embed multiple piezoelectric sensors into PDMS (FIGS. 5A-C) to gather preliminary data. The piezoelectric sensors can be attached to an amplification board and can be connected to an Arduino board. The three sensors can be placed longitudinally, and their respective positions may be denoted as top, middle, and bottom (e.g., distally, closest to the foot). The data outputted from the piezoelectric sensors and Arduino board can be recorded using MATLAB (MathWorks Inc., Natick, MA, USA). The preliminary TDP cuff prototype used a custom in MATLAB script to record data using a subject's calf (no DVT/PTS), and the sensor data is shown in (FIGS. 5C-E). The TDP cuff was pressurized to 140 mmHg with the calf elevated parallel to the ground in a sitting position, the calf was flexed twice, and the pressure was released slowly. There were several distinct peaks during calf flexing and a signature waveform after the pressure was released. The signature waveform can represent the blood refilling the vein, and the latent period between the top and middle peaks may denote the delay of refilling due to lower position of the sensor.

The TDP cuff may collect data from these different patient groups, such as: control (no DVT/PTS, n=20), DVT (n=40), and PTS group (n=40). Both the DVT and PTS group may have already been diagnosed with their respective condition. The TDP cuff may be placed on a subject's calf or upper thigh to record signals for several minutes (e.g., both static and dynamic). For the static case, the patient may raise their leg to an elevated position and the data may be recorded while compressing and releasing the pressure cuffs on the afflicted area on three separate occasions. The dynamic case may occur as the patient walks, the pressure cuff may compress and release while simultaneously recording data from the device. The sensor data may be saved on a memory module and transferred for signal processing during.

Analog voltage signals may be retrieved from the sensor cuff device for the control, DVT, and PTS groups. The analog voltage signals may be rescaled to a 10-bit scale, the working resolution for the Arduino microcontroller (e.g., 5 volts is an analog value of 1024). As the pressure cuff compresses and releases, the signals from sensors positioned at various levels axially may record a latent period between the peak signal (FIG. 5B) indicating when blood is being pushed or reintroduced into the vein. To quantify the signature acoustic waveform received, an nth order Fourier series may be used to mimic the periodic signal produced from the compression and release cycles initiated by the cuff (e.g., the coefficients from the Fourier series can provide the definition to reproduce the waveform). Also, the time delay or latent period between peaks may be recorded along with the peak intensity for additional metrics to be compared statistically. The post-processed signals may be compared statistically using a three-way ANOVA to determine whether the base signals can discern the diagnosis of DVT/PTS reliably. Lastly, clinical data and the processed signal data may be combined to statistically determine whether a difference can be found between the control−DVT, control−PTS, and DVT−PTS groups.

Ultrasound of subjects may be performed, and standard analysis of ultrasound may be used to measure the compliance and by calculating the incremental diameter and pressure changes in the deep veins of the limb/leg through the time-spaced ultrasound data. These results may be used to confirm that the device is reading distinct signature waveforms from each subject group that corresponds to ultrasound results or measurements. The results of the device and ultrasound may be compared using linear regression techniques to remap the signature waveform data with ultrasound compliance data.

It is expected to the signals from the TDP cuff may be validated or related with the ultrasound imaging data. The validated relationship between acoustic signal may relate to the compliance that is measured. This may allow for the TDP cuff to assess potential differences between DVT/PTS potentially eliminating the need for ultrasound imaging. The validation of this device would provide data inputs used in Aim 2 for the training of various machine learning models that is paired with clinical data. Additionally, the results can be statistically compared using multiple regression of each patient's signal from the device compared to their ultrasound reading. Using regression techniques, a trend of where healthy patients land may be determined to be different from patients with DVT/PTS and there may be a stochastic technique from the results of the device that may allow the diagnosis of patients without the required imaging.

The TDP cuff measures acoustic signals through various layers of tissues, muscles, and arteries/veins. There may be an effective range of detecting the signature waveforms depending on the circumference of a patient's calf or thigh or other limb region. Additional operational amplifiers paired with bandpass filters may be used to enhance acoustic signals from the TDP cuff. Depending on the signal-to-noise ratio, the signature waveforms may be normalized to the peak intensity or from anthropometric data (e.g., body mass index, weight, or height). Another potential pitfall may arise from the ultrasound images of DVT and PTS patients and their ability to distinguish differences in compliance. If this is the case, signals extracted spatially from the TDP cuff may be used to statistically compare responses independent of the ultrasound imaging data. Additionally, the signals from the TDP cuff can be extrapolated to measure the functionality between normal, DVT, and PTS patients (e.g., ultrasound imaging is not a requirement, but compliance may not be included) to extrapolate the relationship.

The TDP cuff developed from the previous aim records a stream of data from piezoelectric sensors to assess venous function. A signal processor is used to relay the information provided by the sensors to the severity of diagnosis of DVT or PTS. Additional signal processing and pre-processing of data may be performed to provide inputs to an ML model for classification and risk assessment of predictive disease state (e.g., PTS). The signal processing from the cuff may allow for discernment of the disease state, however, additional inputs from the patient's electronic health record and processed signals from the TDP cuff may allow for additional striation of the patient outcome. Therefore, an ML approach may aid in the validation and development of a software-based tool to process data and provide a predictive assessment and diagnosis of the disease state.

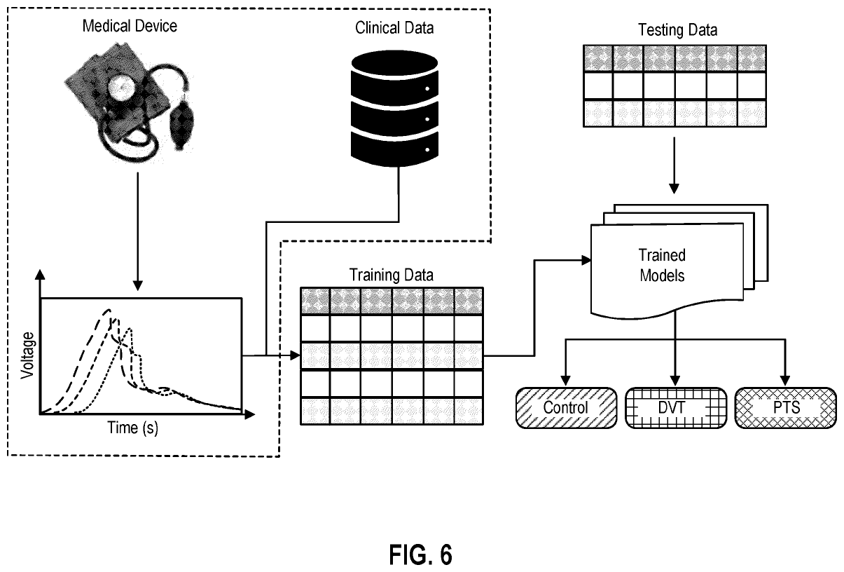

The functional TDP cuff medical device prototype from Aim 1 may be used to record data from the different patient groups (e.g., control, DVT, and PTS). There may be a range of ML models trained that accommodate the different data types that are available. These data types may include clinical data, diagnosis of DVT or PTS, compliance measurements from ultrasound, and TDP cuff data. An ML model may be trained using inputs that were tabulated (TDP cuff data and clinical data) to striate patient outcomes. A separate ML model may be trained using data from ultrasound and clinical data to determine whether the current clinical imaging standard (ultrasound) can be used to develop a predictive model. FIG. 6 provides an example overview of the data sources from the medical device and clinical data that are used for statistical analyses and training/testing machine learning models.

The clinical dataset may be prepared for the control, PTS, and DVT groups by tabulating demographic information, co-morbidities, and/or pharmaceutical use. The data may be prepared by encoding binary variables as ‘0’ or ‘1’ where applicable or expanding to additional integers when two or more unique data points in a category are available (e.g., race may have more than two options). Patient outcomes may be encoded using ‘0’ for the control groups, ‘1’ for DVT, and ‘2’ for PTS. An ML multi-classification model may be trained with the tabulated variables from the clinical data and post-processed data signals using python libraries (sci-kit learn), XGBoost, and algorithms within the MATLAB classification library. The training dataset may include 75% of the patient data that is collected from the pressure cuff and clinical data, and the testing dataset may include the remaining 25% of the patients. Several models may be trained using clinical data alone, TDP cuff acquired data, and/or a combined dataset. The highest internal cross-validation model may be chosen as the model used to striate patient outcomes. Model refinement steps may occur by dimensionality reduction using Gini feature importance to rank variables that are most important to each model and truncated the number of variables. The trained models may be tested by inputting the testing dataset (e.g., 25% of the patient cohort) to predict the diagnosis of each patient. In some embodiments, the models may be assessed based on the composition of signal processed data and clinical data.

The approach may be mirrored to train, test, and validate a machine learning model, however, ultrasound data may be used in place of the TDP cuff data. The motivation behind this approach is to compare the predictability of the current medical imaging standard with the newly developed hardware and software approach. A goal is to assess the utility of the TDP cuff and trained ML model comparing it to the ultrasound trained ML model. This may serve as a validation step for the hardware device to demonstrate that the data acquired from the TDP cuff may be requisite/useful for developing high-accuracy predictive models. In some embodiments, this approach using ultrasound data could expedite a software tool to predict the transition from DVT to PTS using ultrasound and clinical data in a clinical setting.

Pipelines may be used to quickly convert clinical datasets into an encoded table from previous studies using electronic health records. Additionally, the approach can include performing various levels of signal processing that include fitting various types of Fourier series to waveforms. ML training pipelines may be used to train, test, and assess multi-classification models using various automation scripts. An approach can include using AutoML where hyperparameters are automatically perturbed to extract the highest performing trained model (e.g., having best internal cross-validation score).

It is expected that an ML classification model may be used to assist the TDP cuff to accurately predict patients that may transition from DVT to PTS. The classification model may have a truncated list of variables that weigh into the overall weights of each model to predict the diagnosis of each patient. Furthermore, traditional statistical approaches may be used to distinguish signals that may be different between the control and DVT/PTS groups that the medical device could use as a standalone device (e.g., with no additional inputs from clinical data).

A statistical methods may sufficiently determine the differences between control and DVT/PTS groups. However, the ML classification models can serve as a basis for striating patient diagnosis with higher accuracy. The proposed ML model using ultrasound and clinical data may not provide accurate predictions of patients who may develop PTS. This may be likely as ultrasound as the current clinical standard cannot independently diagnose or predict the transition. There may be an insufficient amount of data to reliably train various ML models (e.g., depending on the amount of data that can be collected from patients). To counter this potential pitfall, data augmentation methods may be used to expand the number of patient's data used to train the ML models.

The initial results and validated prototype of the medical device and ML approach to accurately predict the transition from DVT to PTS may provide the basis for follow on towards a commercialization pathway. The overall approach of the sensor cuff and ML may allow clinicians to non-invasively assess the severity of the disease state in static and dynamic scenarios. This approach is lower cost than the current clinical standards and may allow accessibility to reduce healthcare disparities that arise in minority communities where DVT and PTS are most prevalent. Further, if this device is successful in predicting PTS, the technique could be repurposed to other parts of the body where venous abnormalities arise.

### C. Systems and Methods for Determining Likelihood of Vascular Conditions Using Acoustic Measurements of Veins in Limbs of Subjects

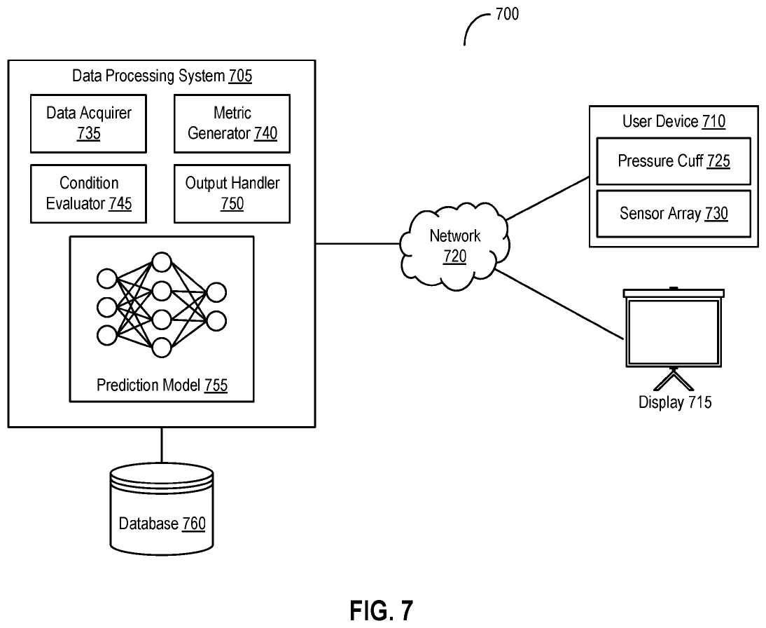

Referring now to FIG. 7 depicts a block diagram of a system 700 for determining likelihood of vascular conditions using acoustic measurements of veins in limbs of subjects. In brief overview, the system 700 may include at least one data processing system 705, at least one user device 710, and at least one display 715, communicatively coupled with one another via at least one network 720. The user device 710 may contain, house, or otherwise include at least one pressure cuff 725 and at least one sensor array 730, among others. The data processing system 705 may include at least one data acquirer 735, at least one metric generator 740, at least one condition evaluator 745, at least one output handler 750, at least one prediction model 755, and at least one database 760, among others. Each of the components in the system 700 as detailed herein may be implemented using hardware (e.g., one or more processors coupled with memory), or a combination of hardware and software as detailed herein in Section D. Each of the components in the system 700 may implement or execute the functionalities detailed herein, such as those described in Sections A and B.

In further detail, the data processing system 705 may be any computing device comprising one or more processors coupled with memory and software and capable of performing the various processes and tasks described herein. The data processing system 705 may be in communication with user device 710 and the display 715 the network 720. The data processing system 705 may be situated, located, or otherwise associated with at least one computer system. The computer system may correspond to a data center, a branch office, or a site at which one or more computers corresponding to the data validation system 705 are situated.

Within the data processing system 705, the data acquirer 735 may retrieve any information (e.g., acoustic measurement, clinical information, and survey data) about a subject to be examined for vascular conditions or disease. The metric generator 740 may determine a set of metrics using the information retrieved about the subject. The condition evaluator 745 may determine a likelihood of a presence or absence of the vascular conditions in the subject using the set of metrics. The output handler 750 may generate an output based on the determined likelihood and other information about the subject. The prediction model 755 may include a machine learning (ML) model or a statistical model (or any combination thereof), and may be used to determine the likelihood (e.g., probability, extent, severity) of the vascular conditions in subjects. The ML model may include, for example, a deep learning artificial neural network (ANN), Naïve Bayesian classifier, a relevance vector machine (RVM), or a support vector machine (SVM), among others. The statistical model may include, for example: a regression model (e.g., linear or logistic regression) or a clustering model (e.g., k-NN clustering or density-based clustering), or a decision tree (e.g., a random tree forest), among others. The database 760 may store and maintain data in connection with the vascular conditions in subjects.

The user device 710 may be any computing device comprising one or more processors coupled with memory and software and capable of performing the various processes and tasks described herein. The user device 710 may be in communication with the data processing system 705 and the display 715 via the network 720, for example, to send data associated with the pressure cuff 725 and the sensor array 730. The user device 710 may be a smartphone, other mobile phone, tablet computer, wearable computing device (e.g., smart watch, eyeglasses), or laptop computer. In some embodiments, the user device 710 may be a wearable device (e.g., a wearable activity tracker) capable of being fitted to a part of a body of a user (e.g., a region on a limb). In some embodiments, the user device 710 may include some or all of the functionalities ascribed to the data processing system 750, including the data acquirer 735, the metric generator 740, the condition evaluator 745, the output handler 750, the prediction model 755, and the database 760. In some embodiments, the functionalities may be shared between the user device 710 and the data processing system 705.

In the user device 710, the pressure cuff 725 (sometimes herein referred to as a pressurizer or a compression device) may provide, produce, or otherwise apply pressure to at least a portion of a limb (e.g., a leg or an arm or a portion thereof) of a subject. In applying pressure, the pressure cuff 725 may control, prevent or interrupt blood flow within veins, arteries, or capillaries within the limb of the subject for a period of time. In some embodiments, the pressure cuff 725 may apply pressure radially about the limb. For example, the pressure cuff 725 may be an intermittent pneumatic compression device to apply inward, pneumatic pressure around the limb. The pressure cuff 725 may contain, house, or otherwise include at least one cuff, at least one inflator, and at least one pressure controller. The cuff may be secured or wrapped at least partially around the limb. The cuff may include a chamber (e.g., an air bladder) to be inflated to apply the pressure to the limb. The inflator may include at least one pump to input or add gas (e.g., air) into the chamber of the cuff. The pressure controller may control, regulate, or otherwise handle (e.g., using one or more processors and memory) the inflation of the gas from the inflator into the chamber of the pressure cuff.

In addition, the sensor array 730 may include a set of acoustic sensors to obtain or acquire acoustic measurements from the limb of the subject on which the pressure cuff 725 is to apply pressure. The acoustic measurement may originate from within the limb, through one or more layers of tissue, muscles, arteries, or veins in the limb of the subject, as the pressure cuff 725 is released. The acoustic measurements may be in a frequency range between 0 Hz and 20 kHz (e.g., below ultrasonic). In some embodiments, the set of acoustic sensors in the sensory array 730 may be acoustoelectric transducer to acquire the acoustic measurements from the limb to convey as electrical signals. Each acoustoelectric transducer may be, for example, a microphone, such as a capacitor microphone, a direct current (DC) condenser microphone, or an electret microphone, among others.

In some embodiments, the set of sensors of the sensor array 730 may be piezoelectric sensors. Each of the piezoelectric sensors may acquire acoustic measurements corresponding to changes in pressure (or acceleration, temperature, strain, or force) in the limb, and convert the measurements to electrical signals (e.g., via a piezoelectric effect). Each piezoelectric sensor may include at least one piezoelectric element (e.g., crystal such as quartz, piezoelectric ceramic such as lead zirconate titanate (PZT), or piezoelectric polymer) to create an electric charge in response to mechanical stress or pressure, and one or more electrodes to collect the charge created by the piezoelectric element.

The display 715 can be communicatively coupled with the data processing system 705 or any other computing device comprising one or more processors coupled with memory and software and capable of performing the various processes and tasks described herein. The display 715 may display, render, or otherwise present any information provided by the image processing system 705 or information provided by the user device 710, or both. The information may be used by a clinician (e.g., a doctor or nurse) examining a subject to define an administration of a treatment to the subject. In some embodiments, the display 715 may be part of the data processing system 705 or may be part of the user device 710. In some embodiments, the display 715 may be with a computing device separate from the data processing system 705 and the user device 710.

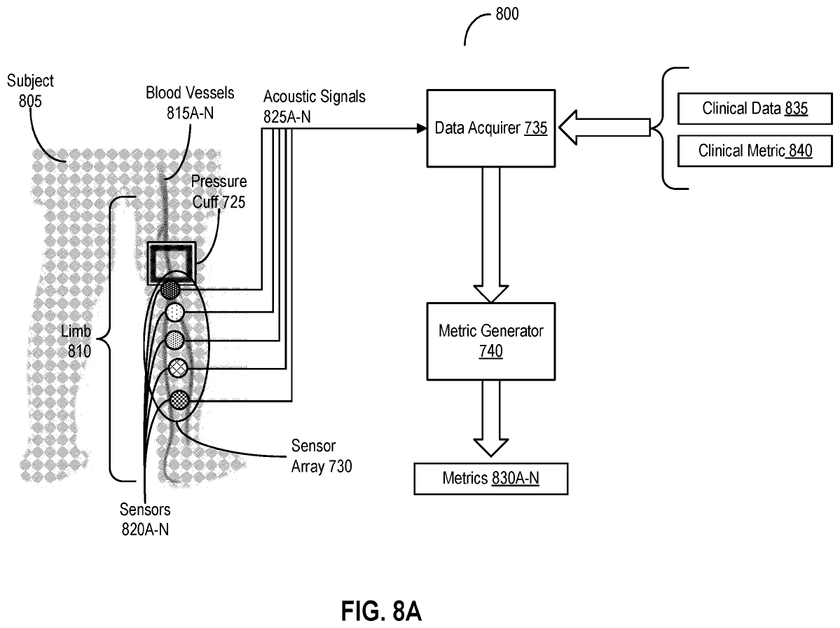

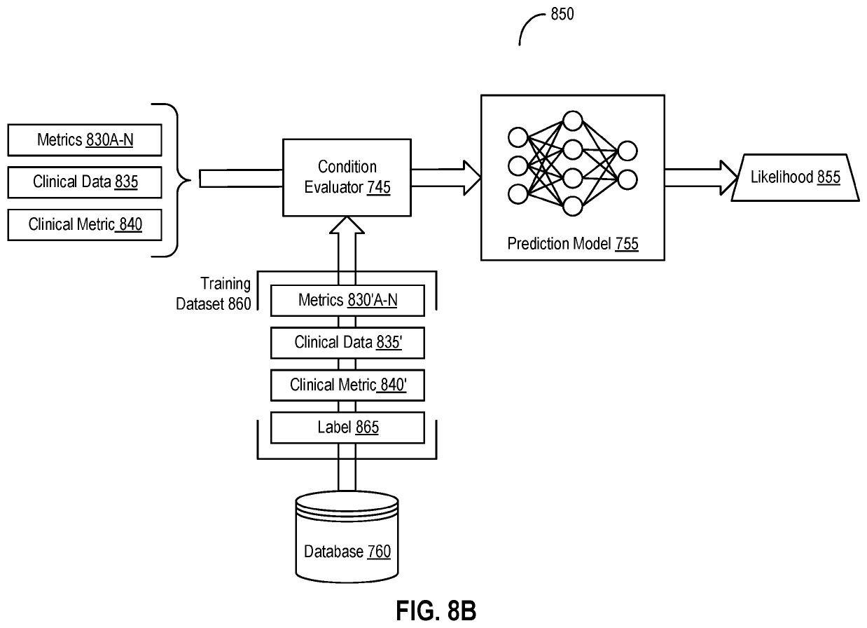

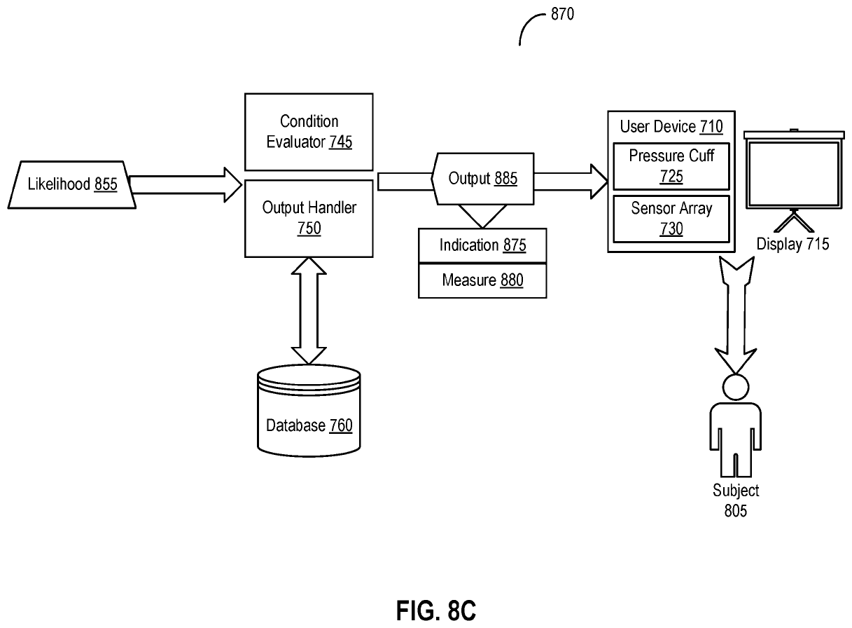

Referring now to FIG. 8A, depicted is a block diagram of a process 800 to acquire acoustic (e.g., vibrational, bio-mechanical, fluidic flow, frictional) measurements and derive metrics in the system 700 for determining likelihood of vascular conditions. The process 800 may include or correspond to operations in the system 700 to acquire acoustic data and generate metrics. Under the process 800, the data acquirer 735 executing on the data processing system 705 may retrieve, identify, or otherwise receive data associated with at least one subject 805. The data acquirer 735 may communicate with the user device 710 (including the pressure cuff 725 and the sensory array 730) to exchange data associated with the subject 805. The subject 805 may be a human or an animal at risk of, to be evaluated for, or suffering from a vascular condition or disease. The vascular condition may include, for example: deep vein thrombosis (DVT); post thrombotic syndrome (PTS), chronic venous insufficiency, peripheral vascular disease, limb ischemia, phlebitis, thromboangiitis obliterans, or lymphedema, among others.

The subject 805 may have at least one limb 810 from which at least a portion of the data is obtained to evaluate for the vascular condition. The limb 810 may be, for example, a left arm, a right arm, left leg, or a right leg, of the subject 805. When the limb is one of the arms, the limb 810 may include an upper arm, a joint (e.g., elbow) and a lower arm (also referred herein as a forearm), among others. In general, the upper arm may correspond to a portion of the arm between a shoulder and the joint. The lower arm may correspond to a portion of the arm between the joint and a hand. When the limb is one of the legs (e.g., as depicted), the limb 810 may include an upper leg (e.g., buttock and thigh), a joint (e.g., knee), and a lower leg (e.g., calf, shin, or ankle), among others. In general, the upper leg may correspond to a portion of the leg between the hip and the joint. The lower leg may correspond to a portion of the leg between the joint and the ankle of the feet of the subject 805.

The limb 810 may contain, house, or otherwise have one or more blood vessels 815A-N (hereinafter generally referred blood vessels 815). At least some of the blood vessels 815 may be at risk of, afflicted with, evaluated for, or may have the vascular condition. The blood vessels 815 may extend or span at least partially through the limb 810. The blood vessels 815 may include, for example, at least one vein to return deoxygenated blood from the limb 810 toward a heart; at least one artery to carry oxygenated blood from the heart through the limb 810; or at least one capillary to convey blood between the vein and artery, among others. The vein and artery may be larger in diameter than the capillary. The limb 810 may also include one or more layers of fat, muscle, tissue, and other organs of the subject 805. The layers may encompass, cover, or otherwise surround the one or more blood vessels 815 in the limb 810.

The pressure cuff 725 may be positioned, attached, or otherwise secured on at least one region of the limb 810 of the subject 805. In the depicted example, the pressure cuff 725 may be fixed, clasped, or otherwise fastened by the subject 805 (or a clinician examining the subject 805) around the thigh region of the leg. The pressure cuff 725 may be secured on the region of the limb 810 using at least one fastening element. The fastening element may include, for example: a strap, a buckle, a hook and loop ring, a snap fastener, elastic band, or a cord, among others. The pressure cuff 725 may produce, output, or otherwise apply pressure to the region of the limb to compress one or more blood vessels 815 for a period of time. The period of time may range, for example, between 15 seconds to 90 seconds. Subsequent to applying the pressure for the period of time, the pressure cuff 725 may cease, discontinue, or otherwise release the application of the pressure on the region of the limb 810 of the subject 805. The pressure cuff 725 may repeat the application and then the releasing of the pressure any number of times to facilitate the measurement of the blood flow by the sensor array 730.

The sensor array 730 may be situated, placed, or otherwise positioned on the region of the limb 810 of the subject 805. The sensor array 730 may be positioned on a portion of the limb 810 further away from the heart of the subject 805 relative to a portion of the limb 810 to which the pressure cuff 725 is secured. In the depicted example, as the calf region of the leg is further away from the heart than the thigh region, the sensor array 730 may be positioned on the calf region below the thigh region where the pressure cuff 725 is secure. In some embodiments, the sensor array 730 may be positioned, attached, or otherwise secured on the region of the limb 810. The sensor array 730 may be secured about the region of the limb 810 using a fastening element, such as a strap, a buckle, a hook and loop ring, a snap fastener, elastic band, or a cord, among others.

The sensor array 730 may contain, house, or otherwise include a set of sensors 820A-N (hereinafter generally referred to as sensors 820). The set of sensors 820 may be situated, arranged, or positioned on the region of the limb 810. In some embodiments, at least a portion of the set of sensors 820 may be arranged radially across the region of the limb 810. For example, two or more of the sensors 820 may be situated on the positions along a latitudinal axis along a width of the leg of the subject 810. The latitudinal axis across the width of the leg may be substantially parallel (e.g., within 80%) to a ground on which the subject 805 is standing. In some embodiments, at least a portion of the set of sensors 820 may be arranged longitudinally on the region of the limb 810. For instance, as in the depicted example, two or more of the sensors 820 may be arranged lengthwise along a longitudinal axis the calf region of the leg. The longitudinal axis across the length of the leg may be substantially orthogonal (e.g., within 80%) to a ground on which the subject 805 is standing. The positions of the set of sensors 820 relative to the region of the limb 810 may be longitudinal or latitudinal, among others or any combination thereof.

The set of sensors 820 may obtain, receive, or otherwise acquire acoustic measurements from one or more vascular pathways or channels, such as one or more blood vessels 815 in the limb 810 of the subject 805. The acoustic measurement may originate from within the limb 810, and may traverse through one or more layers of tissue, muscles, or blood vessels 815 in the limb 810 of the subject 805. The acoustic measurements may be in a frequency range between 0 Hz and 20 kHz (e.g., below ultrasonic). As discussed above, the set of sensors 820 in the sensor array 730 may include, for example: acoustoelectric transducers to acquire the acoustic waves from the limb 810 to convey as electrical signals; or piezoelectric sensor to acquire acoustic measurements corresponding to changes in pressure (or acceleration, temperature, strain, or force) in the limb 810, among others.

In the sensor array 730, the set of sensors 820 may produce, output, or otherwise generate a corresponding set of acoustic signals 825A-N (hereinafter generally referred to as acoustic signals 825). Each sensor 820 may obtain, receive, or otherwise acquire a respective acoustic signal 825 from the one or more blood vessels 815 in the limb 810. The sensor 820 may acquire the acoustic signal 825 as the pressure cuff 725 applies pressure and then releases the pressure, repeatedly. The respective acoustic signal 825 may correspond to an acoustic measurement (e.g., acoustic wave or change in pressure) at the respective sensor 820. Upon receipt of the acoustic measurement, the sensor 820 may transform or convert the acoustic measure to an electrical signal corresponding to the acoustic signal 825. The conversion may be performed using the acoustic transducer or the piezoelectric sensor of the sensor 820. Each acoustic signal 825 may be in a time domain. For example, the acoustic signal 825 may be a time-series data generated from the conversion of the sound waves or pressure changes to electric signals.

Continuing on, in some embodiments, the set of sensors 820 may generate the set of acoustic signals 825, in response to the pressure cuff 725 releasing the pressure to the region of the limb 810. For example, the processors on the user device 710 may detect the releasing or easing of the pressure through the pressure cuff 725, and may activate the sensor array 730 to initiate acquisition of the set of acoustic sensors 825 through the set of sensors 820. With the acquisition, the set of sensors 820 may send, forward, or otherwise transmit the set of acoustic signals 825 to the data acquirer 735. As each sensor 820 acquires additional acoustic measurements, the sensor 820 may continue to transmit the acoustic signal 825 to the data acquirer 735. Each sensor 820 may transmit the respective acoustic signal 825 for a period of measurement time subsequent to the pressure cuff 725 releasing of the pressure on the region of the limb 810. The period of time may range, for example, from 1 minute to 10 minutes. In some embodiments, each sensor 820 may transmit the respective acoustic signal 825 to the data acquirer 735 subsequent to the period of measurement time. In some embodiments, each sensor 820 may generate the set of acoustic signals 825 while the pressure cuff 725 applies the pressure and then releases the pressure, repeatedly. The sensor 820 may transmit the respective acoustic signal 825 for a period of measurement time corresponding to the application and releasing of the pressure by the pressure cuff 725 over multiple times.

The data acquirer 735 may retrieve, identify, or otherwise receive the set of acoustic signals 825 from the set of sensors 820 of the sensor array 730. In some embodiments, the data acquirer 735 may check or monitor whether the pressure cuff 725 is applying or has released the pressure on the region of the limb 810 of the subject 805. When the pressure cuff 725 is applying the pressure, the data acquirer 735 may continue to monitor until detecting that the pressure cuff 725 has released the pressure. In some embodiments, the data acquirer 735 may receive the set of acoustic signals 825 during the application and releasing of the pressure. With the detection of the releasing of the pressure, the data acquirer 735 may initiate receipt of the set of acoustic signals 825 from the set of sensors 820. In some embodiments, the data acquirer 735 may enable or activate the set of sensors 820 in the sensor array 730 to initiate acquisition of the set of acoustic signals 825 in response to the pressure cuff 725 releasing the pressure on the region of the limb 810. The set of acoustic signals 825 may be over the measurement time period. Each acoustic signal 825 may correspond to a time-domain measurement corresponding to sound waves or changes in pressure. These measurements may result from the reintroduction of the blood flow within a portion of the blood vessels 815 relative to the position of the respective sensor 820 positioned on the limb 810, upon releasing of the pressure. Each acoustic signal 825 may correspond to a combination of set of periodic signals originating the blood flow in the blood vessels 815, due to the reintroduction of the blood flow.

In some embodiments, the data acquirer 735 may combine or aggregate one or more of the set of acoustic signals 825 (or measurements corresponding to the signals 825) in accordance with a respective position of each sensor 820 on the region of the limb 810. The aggregation based on arrangement of the sensors 820 may factor in delays or latency due to differences in positions of the sensors 820 relative to the pressure cuff 725 on the limb 810. For example, as the pressure cuff 725 eases pressure on the limb 810, the resultant set of acoustic signals 825 may have different peaks corresponding to when the blood is reintroduced or pushed into the portion of the blood vessels 815 at the respective sensors 820 on the region of the limb 810. The data acquirer 735 may aggregate subsets of acoustic signals 825 based on a position (e.g., latitudinal position) on which the respective sensors 820 are arranged.

To aggregate, the data acquirer 735 may identify the position of the respective sensor 820 from which the corresponding acoustic signal 825 originates. With the identifications, the data acquirer 735 may find or identify at least one other sensor 820 on the same axis (or similar relative distance from the pressure cuff 725) as part of the same subset, and may select or identify the other acoustic signal 825 originating from the other sensor 820. The data acquirer 735 may combine or aggregate the acoustic signals 825 on the same axis or similar relative distance (e.g., within 90%) to generate at least one acoustic signal 825 to use for the subset of sensors 820. For example, the data acquirer 725 may add and then normalize the acoustic signals 825 from the identified subset of sensors 820 of the sensor array 730. In some embodiments, the data acquirer 735 may apply pre-processing on the set of acoustic signals 825, prior to analysis. The pre-processing may include, for example, smoothing algorithm (e.g., using a low-pass filter, such as Kalman filter, Butterworth filter, Chebyshev filter, and Bessel filter) or a noise reduction (e.g., dynamic noise reduction), among others.

The metric generator 740 executing on the data processing system 705 may calculate, determine, or otherwise generate a set of metrics 830A-N (hereinafter generally referred to as metrics 830) using the set of acoustic signals 825. The set of acoustic signals 825 may be time domain measurements of the sound waves or changes in pressure within the blood vessels 815 in the limb 810 of the subject 805. The set of metrics 830 may correspond to or identify a representation of the corresponding set of acoustic signals 825. For each acoustic signal 825, the metric generator 740 may derive or generate one or more metrics 830. The metrics 830 may be used to evaluate vascular conditions in the blood vessels 815 of the limb 810 of the subject 805.

To generate, the metric generator 740 may alter, transform, or otherwise convert each acoustic signal 825 from the time domain to a frequency domain to generate one or more corresponding metrics 830 for the set of metrics 830. The conversion may be in accordance with a Fourier or other transformation (e.g., a discrete-time Fourier transform (DTFT), a short-time Fourier transform (STFT), or a discrete cosine transform (DCT)), a Hilbert transform, a wavelet transformation, Constant-Q transform (CQT), or a Z-transform, among others. Each metric 830 may represent, define, or otherwise identify a corresponding coefficient in the frequency domain from the conversion of the time-domain series in the set of acoustic waves 825. For example, each metric 830 may identify a Fourier coefficient (e.g., from DTFT, STFT, or DCT). The Fourier coefficients corresponding to the set of metrics 830 may quantify or define the set of periodic waveforms constituting the given acoustic signal 825. The periodicity of the waveforms corresponding to the acoustic signal 825 and by extension the Fourier coefficients corresponding to the metrics 830 may be the result of the blood flow due to the compressing and releasing of the pressure by the pressure cuff 725.

In some embodiments, the metric generator 740 may generate the set of metrics 830 from the set of acoustic signals 725 according to curve fitting. The curve fitting may include, for example, a polynomial interpolation, a geometric interpolation, or function approximation, among others. For each acoustic signal 725, the metric generator 740 may apply curve fitting to derive or generate one or more metrics 830. Each metric 830 may represent, define, or otherwise identify a corresponding coefficient for the curve fitting of the acoustic signal 725. For instance, the metric 830 may be a corresponding n-th coefficient for a polynomial curve approximating the acoustic signal 725.

In some embodiments, the data acquirer 735 may retrieve, identify, or otherwise receive clinical data 835 associated with the subject 805. The clinical data 835 may include or identify information about the subject 805, such as: demographic information (e.g., age, gender, location, and race); usage of a treatment such as a pharmaceutical (e.g., heparin, warfarin, apixaban or anticoagulant for DVT) or other intervention (e.g., compression therapy); or at least one co-morbidity identifying a presence of condition, disease, or risk factor (e.g., cardiac, renal, hematological, rheumatological, gastrointestinal, respiratory, endocrine conditions, or infections) or in the subject 805, among others, or any combination thereof. In some embodiments, the metric generator 740 may add, insert, or otherwise include the clinical data 835 with the set of metrics 830 to be used to evaluate the subject 805 for the vascular condition.

In some embodiments, the data acquirer 735 may retrieve, identify, or otherwise receive at least one clinical metric 840 associated with the subject 805. The clinical metric 840 may be provided (e.g., inputting via the user device 710 or another computing device) by the subject 805 from a survey regarding the vascular condition. For instance, the clinical metric 840 may be based on a self-reported metric in response to the survey presented to the subject 805, such as a Villalta score used to diagnose a presence of PVT in the blood vessels 815 in a given subject. Other metrics that can be used for the clinical metric 8404 may include, for example, Well's criteria, Caprini score, or D-Dimer test, among others. In some embodiments, the metric generator 740 may add, insert, or otherwise include the clinical metric 840 with the set of metrics 830 to be used to evaluate the subject 805 for the vascular condition.

Referring now to FIG. 8B, depicted is a block diagram of a process 850 to determine vascular condition likelihoods in the system 700 for determining likelihood of vascular conditions. The process 850 may include or correspond to using data associated with a given subject to determine a likelihood of a vascular condition in the subject. Under the process 850, the condition evaluator 745 executing on the data processing system 705 may calculate, generate, or otherwise determine at least one likelihood 855 using the data associated with the subject 805. The likelihood 855 may identify or indicate a value for a presence (or absence) of the vascular condition in the one or more blood vessels 815 in the limb 810 of the subject 805. In general, the greater the value of the likelihood 855, the higher the probability that the one or more blood vessels 815 in the limb 810 of the subject 805 may be afflicted or may have the vascular condition. Conversely, the lower the value of the likelihood 855, the lower the probability that the one or more blood vessels 815 in the limb 810 of the subject 805 may be afflicted or may have the vascular condition. In some embodiments, the condition evaluator 745 may generate a set of likelihoods 855 for a corresponding set of vascular conditions. Each likelihood 855 identify the value for the presence (or the absence) of the respective vascular condition in the blood vessels 815 (e.g., a vein) in the limb 810 of the subject 805.

To determine the likelihood 855, in some embodiments, the condition evaluator 745 may use the set of metrics 830, the clinical data 835, or the clinical metric 840, or any combination thereof, among others. In some embodiments, the condition evaluator 745 may determine the likelihood 855 based on the set of metrics 830. In some embodiments, the condition evaluator 745 may determine the likelihood 855 based on the set of metrics 830, along with at least one of the clinical data 835 or the clinical metric 840, or both. In some embodiments, the condition evaluator 745 may determine the likelihood 855 in accordance with a function. In some embodiments, the function may identify, define, or otherwise a mapping between the set of metrics 830 (along with the clinical data 835 or the clinical metric 840 or both) with the value for the likelihood 855. In some embodiments, the function may specify a weighted combination of values of the set of metrics 830 (along with the clinical data 835 or the clinical metric 840 or both) to calculate the likelihood 855.

In some embodiments, the condition evaluator 745 may use the prediction model 755 to generate the likelihood 855. In some embodiments, the condition evaluator 745 may initialize, train, and establish the prediction model 755 using at least one training dataset 860 to determine the likelihood 860. The training of the prediction model 755 may be in accordance with supervised learning (e.g., as depicted), weakly supervised learning, or unsupervised learning, among others. The training dataset 860 may identify or include a set of examples. In the training dataset 860, each example may identify or include a set of metrics 830′A-N (hereinafter generally referred to as metrics 830′) and at least one label 865. In some embodiments, the example of the training dataset 860 may identify or include clinical data 835′ and at least one clinical metric 840′.

In each example, the set of metrics 830′ may be generated from a set of acoustic signals (in a similar manner as the acoustic signals 825). The acoustic signals may be acquired from blood vessels in a respective limb (e.g., the same limb or another type of limb) of another given subject, upon releasing of pressure applied to the limb. The label 865 may identify or indicate a presence or absence of the vascular condition in the limb of the subject. The clinical data 835′ may identify information about the subject in the example, such as: demographic information; usage of a treatment such as a pharmaceutical or other intervention; or at least one co-morbidity, among others. The clinical metric 840′ may have generated using a survey regarding the vascular condition from the subject. In some embodiments, the example may identify or include data generated or derived from ultrasound imaging of the blood vessels in the respective limb to measure a compliance (e.g., whether the blood is properly transported through the blood vessel. The data may be used by the condition evaluator 745 to generate the label 865, in order to train the prediction model 755.

With the identification of the training dataset 860, the condition evaluator 745 may feed or apply the set of metrics 830′ of each example to the prediction model 755. In some embodiments, the condition evaluator 745 may also apply the set of metrics 830′, along with the clinical data 835′ or the clinical metric 840′ or both, to the prediction model 755. In applying, the condition evaluator 745 may process the input (e.g., the set of metrics 830′, the clinical data 835′, and the clinical metric 840′) in accordance with the weights of the prediction model 755. From processing, the condition evaluator 745 may output, produce, or otherwise generate a likelihood indicating a presence (or absence) of the vascular condition in the blood vessels of the limb in the subject in the example of the training dataset 860.

The condition evaluator 745 may compare the output likelihood from the prediction model 755 with the label 865 of the example from which the input data was used to generate the output. In some embodiments, the condition evaluator 745 may calculate, identify, or otherwise determine an expected output from the data derived from the ultrasound imaging in the example. The data derived from the ultrasound imaging may identify or indicate the presence or the absence of the vascular condition in the limb of the subject in the example. Based on the comparison, the condition evaluator 745 may calculate, generate, or otherwise determine at least one loss metric. The loss metric may identify or correspond to a degree of deviation from the output from the prediction model 755 and the expected indication (e.g., of the presence or absence) as identified in the label 865 The loss metric may be calculated in accordance with any number of loss function, such as a norm loss (e.g., L1 or L2), mean squared error (MSE), a quadratic loss, a cross-entropy loss, and a Huber loss, among others.

Using the loss metric, the condition evaluator 745 may modify, set, or otherwise update the prediction model 755. The updating of weights of the prediction model 755 may be in accordance with an optimization function (or an objective function). The optimization function may define one or more rates or parameters at which the weights of the prediction model 755 are to be updated. For example, the optimization function may be stochastic gradient descent (SGD)) with a set learning rate, a momentum, and a weigh decay for a number of iterations. The updating of the weights of the prediction model 755 may be repeated until convergence. The prediction model 755 may be established to be used to new incoming input, upon completion of the training.

With the establishment, the condition evaluator 745 may feed or apply the set of metrics 830 to the prediction model 755. In some embodiments, the condition evaluator 745 may also apply the set of metrics 830, along with the clinical data 835 or the clinical metric 840 or both, to the prediction model 755. In applying, the condition evaluator 745 may process the input (e.g., the set of metrics 830, the clinical data 835, and the clinical metric 840) in accordance with the weights of the prediction model 755. From processing, the condition evaluator 745 may output, produce, or otherwise generate the likelihood 855 identifying or indicating a presence (or absence) of the vascular condition in the blood vessels 815 of the limb 810 in the subject 805. The vascular condition may be of the same type as the condition in the examples of the training dataset 860.

Referring now to FIG. 8C, depicted is a block diagram of a process 870 to produce outputs in the system 700 for determining likelihood of vascular conditions. The process 870 may include or correspond to operations performed in the system 700 to provide information in connection with the determined likelihood. The condition evaluator 745 may category or classify the subject 805 with respect to the vascular condition based on the likelihood 855. To classify, the condition evaluator 745 may compare the likelihood 855 with a threshold. The threshold may delineate, specify, or otherwise define a value of the likelihood 855 at which to identify the presence or the absence of the vascular condition in the blood vessels 815 in the limb 810 of the subject 805. Based on the comparison, the condition evaluator 745 may write, produce, or otherwise generate at least one indication 875.

If the likelihood 855 satisfies (e.g., greater than or equal to) the threshold, the condition evaluator 745 may determine or identify the presence of the vascular condition in the blood vessels 815 in the limb 810 of the subject 805. The condition evaluator 745 may classify the subject 805 as afflicted with or having the vascular condition. The condition evaluator 745 may generate the indication 875 to identify the presence of the vascular condition. On the other hand, if the likelihood 855 does not satisfy (e.g., less than) the threshold, the condition evaluator 745 may determine or identify the absence of the vascular condition in the blood vessels 815 in the limb 810 of the subject 805. The condition evaluator 745 may classify the subject 805 as not afflicted with or lacking the vascular condition. The condition evaluator 745 may generate the indication 875 to identify the absence of the vascular condition.

In some embodiments, the condition evaluator 745 may store and maintain an association between the subject 805 and the likelihood 855 of the presence (or absence) of the vascular condition. The storage and maintenance of the association may use one or more data structures (e.g., arrays, matrixes, tables, linked lists, stacks, queues, trees, or heaps) on the database 760. In some embodiments, the condition evaluator 745 may store and maintain the association of the subject 805 with the indication 875 to identify the presence of the vascular condition in the blood vessels 815 in the limb 810 of the subject 805. The association may also be with the set of metrics 830, the clinical data 835, and the clinical metric 840, among others.

In some embodiments, the condition evaluator 745 may keep track of or maintain at least one measure 880 of progress (or improvement) of the vascular condition in the blood vessels 815 in the limb 810 of the subject 805. Each time data associated about the subject 805 is retrieved, the metric generator 740 may generate the set of metrics 830 using the set of acoustic signals 825 in a similar manner as described above. In addition, the condition evaluator 745 may determine the likelihood 855 based on the set of metrics 830 (e.g., along with the clinical data 835 and the clinical metric 840). To determine the measure 880, the condition evaluator 745 may retrieve, obtain, or otherwise identify the likelihoods 855 over multiple samplings. For instance, the condition evaluator 745 may fetch the likelihoods 855 for the subject 805 determined over time.

With the identification, the condition evaluator 745 may compare the likelihoods 855 (or the likelihoods 855) over multiple samplings of the data associated with the subject 805 to determine the measure 855. The measure 880 may define, specify, or otherwise identify a value corresponding to a change of the vascular condition in the blood vessels 815 in the limb 810 of the subject 805. The change may be an improvement or a worsening of the vascular condition. For example, the measure 880 may be a moment of the values of likelihoods 855 over time. If the likelihoods 855 increase over time, the measure 880 may indicate a worsening of the vascular condition in the subject 805. Conversely, if the likelihoods 855 decrease over time, the measure 880 may indicate an improvement of the vascular condition in the subject 805.

In some embodiments, the condition evaluator 745 may determine or identify a change in the vascular condition based on the comparison of the likelihoods 855 or the indications 875 over time. If the indication 875 of the most recent sample differs from the indication 875 of a prior sample, the condition evaluator 745 may identify the change in the vascular condition. Otherwise, if the indication 875 of the most recent sample is the same as the indication 875 of a prior sample, the condition evaluator 745 may identify a lack of change in the vascular condition. When integrated with the user device 710, the condition evaluator 745 may provide, send, or transmit the change in the vascular condition in blood vessels 815 in the limb 810 of the subject 805. For example, the condition evaluator 745 may send the indication of the change of the vascular condition to the data processing system 705 or another computing system.

The output handler 750 executing on the data processing system 705 may produce, create, or otherwise generate at least one output 855. The output 855 may identify or include information about the subject 805 in connection with the evaluation for the vascular condition. The output 855 may identify or include the indication 875 to identify the presence or absence of the vascular condition. When the likelihood 885 satisfies the threshold, the output handler 750 may provide the output 855 including the indication 875 to identify the presence of the vascular condition. Conversely, when the likelihood 885 does not satisfy the threshold, the output handler 750 may provide the output 855 including the indication 875 to identify the absence of the vascular condition. In some embodiments, the output 855 may identify or include the measure 880 tracking progress of the vascular condition for the subject 805 over time. The output 855 may identify or include other information about the subject 805, such as the identifier (e.g., user name), the likelihood 855, the clinical data 835, and the clinical metric 840, among others. With the generation, the output handler 750 may send, transmit, or otherwise provide the output 855 to the user device 710, the display 715, or another computing device, among others.

Upon receipt, the user device 710 may store and maintain the output 855 on a local memory. The user device 710 may use the output 855 to compare with future outputs to determine progress and changes with respect to the vascular condition in the blood vessels 815 in the limb 810 of the subject 805. In addition, the display 715 (or a computing device connected thereto) may display, render, or otherwise present the output 855 from the output handler 750. For example, the display 715 may present the indication 875 to identify the presence or absence of the vascular condition in the subject 805 and the measure 880 identifying the progress for the subject 805. The output 855 presented via the display 715 may be used by the clinician examining the subject 805 to diagnose the subject 805 for the vascular condition. Furthermore, the output 855 may be used by the clinician to determine whether to administer treatment for the vascular condition in the blood vessel 815 in the limb 810 of the subject 805.

In this manner, the data processing system 705 may enable use of acoustic signals 825 acquired from the blood vessels 815 within the limb 810 of the subject 805 to derive the likelihood 855 of the presence or absence of the vascular condition. The data processing system 705 may provide for more accurate prediction of the presence or absence of the vascular condition, thereby improving clinical outcomes. The technique may also forego reliance of invasive techniques to draw blood from the subject 805 or ultrasound techniques involving application of ultrasonic energy externally onto the limb 810. As result, the data processing system 705 may reduce or eliminate the reliance on invasive techniques and may lower or decrease the consumption of electric power from the application of the ultrasonic energy.

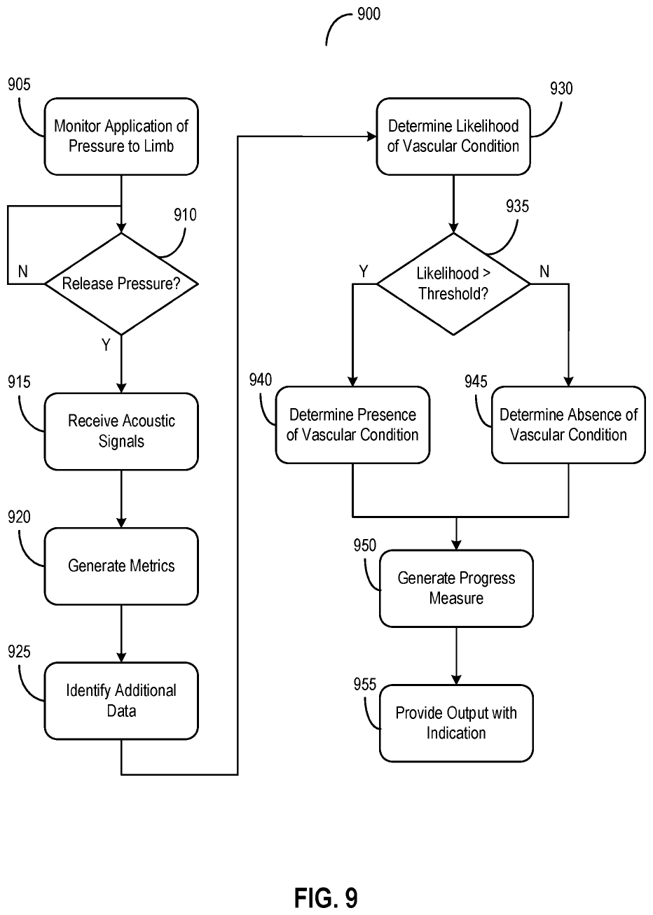

Referring now to FIG. 9, depicted is a flow diagram of a method 900 of determining likelihood of vascular conditions using acoustic measurements of veins in limbs of subjects. The method 900 may be performed by or implemented using the components described in Sections A or B, the system 700 described herein in conjunction with FIGS. 8-9C, or the system 1000 detailed herein in Section D. Under the method 900, a computing system may perform monitoring of an application of pressure to a vein in a limb of a subject (905). The computing system may detect releasing of the pressure on the limb (910). If the releasing of the pressure is detected, the computing system may receive a set of acoustic signals measured on the limb of the subject (915). The computing system may generate a set of metrics using the set of acoustic signals (920). The computing system may identify additional data associated with the subject (925).

Continuing on, the computing system may determine a likelihood of a presence or absence of a vascular condition in the limb of the subject based on the set of metrics and the additional data (930). The computing system may determine whether the likelihood satisfies a threshold (935). If the likelihood satisfies (e.g., greater than) the threshold, the computing system may determine the presence of the vascular condition (940). Conversely, if the likelihood does not satisfy (e.g., less than or equal to) the threshold, the computing system may determine the absence of the vascular condition (945). The computing system may generate a progress measure (950). The computing system may provide an output with an indication (955).

### D. Computing and Network Environment

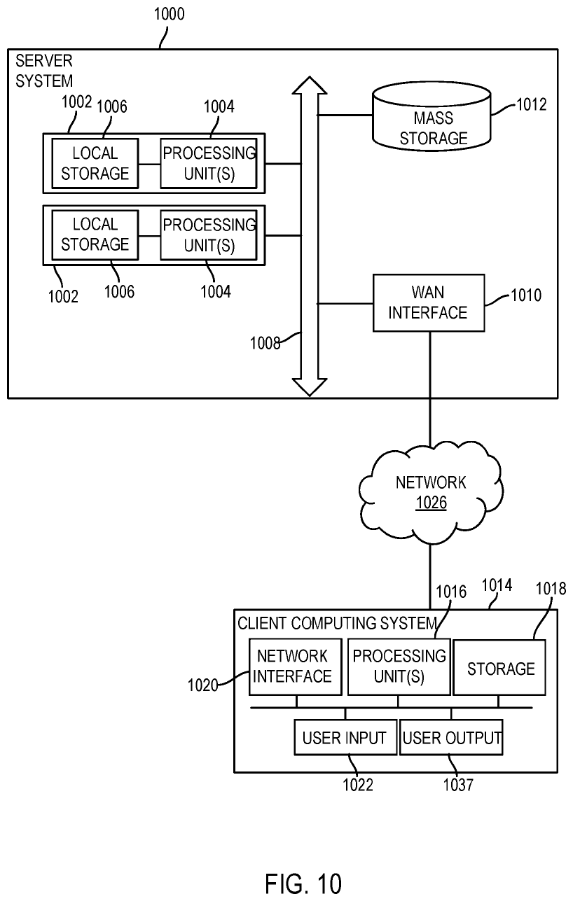

Various operations described herein can be implemented on computer systems. FIG. 10 shows a simplified block diagram of a representative server system 1000, client computing system 1014, and network 1026 usable to implement certain embodiments of the present disclosure. In various embodiments, server system 1000 or similar systems can implement services or servers described herein or portions thereof. Client computing system 1014 or similar systems can implement clients described herein. The system 700 described herein can be similar to the server system 1000. Server system 1000 can have a modular design that incorporates a number of modules 1002 (e.g., blades in a blade server embodiment); while two modules 1002 are shown, any number can be provided. Each module 1002 can include processing unit(s) 1004 and local storage 1006.

Processing unit(s) 1004 can include a single processor, which can have one or more cores, or multiple processors. In some embodiments, processing unit(s) 1004 can include a general-purpose primary processor as well as one or more special-purpose co-processors such as graphics processors, digital signal processors, or the like. In some embodiments, some or all processing units 1004 can be implemented using customized circuits, such as application specific integrated circuits (ASICs) or field programmable gate arrays (FPGAs). In some embodiments, such integrated circuits execute instructions that are stored on the circuit itself. In other embodiments, processing unit(s) 1004 can execute instructions stored in local storage 1006. Any type of processors in any combination can be included in processing unit(s) 1004.

Local storage 1006 can include volatile storage media (e.g., DRAM, SRAM, SDRAM, or the like) and/or non-volatile storage media (e.g., magnetic or optical disk, flash memory, or the like). Storage media incorporated in local storage 1006 can be fixed, removable or upgradeable as desired. Local storage 1006 can be physically or logically divided into various subunits such as a system memory, a read-only memory (ROM), and a permanent storage device. The system memory can be a read-and-write memory device or a volatile read-and-write memory, such as dynamic random-access memory. The system memory can store some or all of the instructions and data that processing unit(s) 1004 need at runtime. The ROM can store static data and instructions that are needed by processing unit(s) 1004. The permanent storage device can be a non-volatile read-and-write memory device that can store instructions and data even when module 1002 is powered down. The term “storage medium” as used herein includes any medium in which data can be stored indefinitely (subject to overwriting, electrical disturbance, power loss, or the like) and does not include carrier waves and transitory electronic signals propagating wirelessly or over wired connections.

In some embodiments, local storage 1006 can store one or more software programs to be executed by processing unit(s) 1004, such as an operating system and/or programs implementing various server functions such as functions of the system 700 of FIG. 7 or any other system described herein, or any other server(s) associated with system 700 or any other system described herein.

“Software” refers generally to sequences of instructions that, when executed by processing unit(s) 1004 cause server system 1000 (or portions thereof) to perform various operations, thus defining one or more specific machine embodiments that execute and perform the operations of the software programs. The instructions can be stored as firmware residing in read-only memory and/or program code stored in non-volatile storage media that can be read into volatile working memory for execution by processing unit(s) 1004. Software can be implemented as a single program or a collection of separate programs or program modules that interact as desired. From local storage 1006 (or non-local storage described below), processing unit(s) 1004 can retrieve program instructions to execute and data to process in order to execute various operations described above.

In some server systems 1000, multiple modules 1002 can be interconnected via a bus or other interconnect 1008, forming a local area network that supports communication between modules 1002 and other components of server system 1000. Interconnect 1008 can be implemented using various technologies including server racks, hubs, routers, etc.

A wide area network (WAN) interface 1010 can provide data communication capability between the local area network (interconnect 1008) and the network 1026, such as the Internet. Technologies can be used, including wired (e.g., Ethernet, IEEE 1002.3 standards) and/or wireless technologies (e.g., Wi-Fi, IEEE 1002.11 standards).

In some embodiments, local storage 1006 is intended to provide working memory for processing unit(s) 1004, providing fast access to programs and/or data to be processed while reducing traffic on interconnect 1008. Storage for larger quantities of data can be provided on the local area network by one or more mass storage subsystems 1012 that can be connected to interconnect 1008. Mass storage subsystem 1012 can be based on magnetic, optical, semiconductor, or other data storage media. Direct attached storage, storage area networks, network-attached storage, and the like can be used. Any data stores or other collections of data described herein as being produced, consumed, or maintained by a service or server can be stored in mass storage subsystem 1012. In some embodiments, additional data storage resources may be accessible via WAN interface 1010 (potentially with increased latency).

Server system 1000 can operate in response to requests received via WAN interface 1010. For example, one of modules 1002 can implement a supervisory function and assign discrete tasks to other modules 1002 in response to received requests. Work allocation techniques can be used. As requests are processed, results can be returned to the requester via WAN interface 1010. Such operation can generally be automated. Further, in some embodiments, WAN interface 1010 can connect multiple server systems 1000 to each other, providing scalable systems capable of managing high volumes of activity. Other techniques for managing server systems and server farms (collections of server systems that cooperate) can be used, including dynamic resource allocation and reallocation.

Server system 1000 can interact with various user-owned or user-operated devices via a wide-area network such as the Internet. An example of a user-operated device is shown in FIG. 10 as client computing system 1014. Client computing system 1014 can be implemented, for example, as a consumer device such as a smartphone, other mobile phone, tablet computer, wearable computing device (e.g., smart watch, eyeglasses), desktop computer, laptop computer, and so on.

For example, client computing system 1014 can communicate via WAN interface 1010. Client computing system 1014 can include computer components such as processing unit(s) 1016, storage device 1018, network interface 1020, user input device 1022, and user output device 1024. Client computing system 1014 can be a computing device implemented in a variety of form factors, such as a desktop computer, laptop computer, tablet computer, smartphone, other mobile computing device, wearable computing device, or the like.

Processing unit(s) 1016 and storage device 1018 can be similar to processing unit(s) 1004 and local storage 1006 described above. Suitable devices can be selected based on the demands to be placed on client computing system 1014; for example, client computing system 1014 can be implemented as a “thin” client with limited processing capability or as a high-powered computing device. Client computing system 1014 can be provisioned with program code executable by processing unit(s) 1016 to enable various interactions with server system 1000.

Network interface 1020 can provide a connection to the network 1026, such as a wide area network (e.g., the Internet) to which WAN interface 1010 of server system 1000 is also connected. In various embodiments, network interface 1020 can include a wired interface (e.g., Ethernet) and/or a wireless interface implementing various RF data communication standards such as Wi-Fi, Bluetooth, or cellular data network standards (e.g., 3G, 4G, LTE, etc.).

User input device 1022 can include any device (or devices) via which a user can provide signals to client computing system 1014; client computing system 1014 can interpret the signals as indicative of particular user requests or information. In various embodiments, user input device 1022 can include any or all of a keyboard, touch pad, touch screen, mouse or other pointing device, scroll wheel, click wheel, dial, button, switch, keypad, microphone, and so on.

User output device 1024 can include any device via which client computing system 1014 can provide information to a user. For example, user output device 1024 can include display-to-display images generated by or delivered to client computing system 1014. The display can incorporate various image generation technologies, e.g., a liquid crystal display (LCD), light-emitting diode (LED) including organic light-emitting diodes (OLED), projection system, cathode ray tube (CRT), or the like, together with supporting electronics (e.g., digital-to-analog or analog-to-digital converters, signal processors, or the like). Some embodiments can include a device such as a touchscreen that function as both input and output device. In some embodiments, other user output devices 1024 can be provided in addition to or instead of a display. Examples include indicator lights, speakers, tactile “display” devices, printers, and so on.

Some embodiments include electronic components, such as microprocessors, storage and memory that store computer program instructions in a computer-readable storage medium. Many of the features described in this specification can be implemented as processes that are specified as a set of program instructions encoded on a computer-readable storage medium. When these program instructions are executed by one or more processing units, they cause the processing unit(s) to perform various operation indicated in the program instructions. Examples of program instructions or computer code include machine code, such as is produced by a compiler, and files including higher-level code that are executed by a computer, an electronic component, or a microprocessor using an interpreter. Through suitable programming, processing unit(s) 1004 and 1016 can provide various functionality for server system 1000 and client computing system 1014, including any of the functionality described herein as being performed by a server or client, or other functionality.

It will be appreciated that server system 1000 and client computing system 1014 are illustrative and that variations and modifications are possible. Computer systems used in connection with embodiments of the present disclosure can have other capabilities not specifically described here. Further, while server system 1000 and client computing system 1014 are described with reference to particular blocks, it is to be understood that these blocks are defined for convenience of description and are not intended to imply a particular physical arrangement of component parts. For instance, different blocks can be but need not be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments of the present disclosure can be realized in a variety of apparatus including electronic devices implemented using any combination of circuitry and software.

While the disclosure has been described with respect to specific embodiments, one skilled in the art will recognize that numerous modifications are possible. Embodiments of the disclosure can be realized using a variety of computer systems and communication technologies including but not limited to the specific examples described herein. Embodiments of the present disclosure can be realized using any combination of dedicated components and/or programmable processors and/or other programmable devices. The various processes described herein can be implemented on the same processor or different processors in any combination. Where components are described as being configured to perform certain operations, such configuration can be accomplished, e.g., by designing electronic circuits to perform the operation, by programming programmable electronic circuits (such as microprocessors) to perform the operation, or any combination thereof. Further, while the embodiments described above may make reference to specific hardware and software components, those skilled in the art will appreciate that different combinations of hardware and/or software components may also be used and that particular operations described as being implemented in hardware might also be implemented in software or vice versa.

Computer programs incorporating various features of the present disclosure may be encoded and stored on various computer-readable storage media; suitable media include magnetic disk or tape, optical storage media such as compact disk (CD) or DVD (digital versatile disk), flash memory, and other non-transitory media. Computer-readable media encoded with the program code may be packaged with a compatible electronic device, or the program code may be provided separately from electronic devices (e.g., via Internet download or as a separately packaged computer-readable storage medium).

Thus, although the disclosure has been described with respect to specific embodiments, it will be appreciated that the disclosure is intended to cover all modifications and equivalents within the scope of the following claims.

We claim:

1. A system for determining likelihood of vascular conditions using acoustic measurements of veins in limbs of subjects, comprising:
a pressure cuff secured about a region of a limb of a subject, the pressure cuff configured to apply pressure to the region to compress at least one vein in the limb of the subject;
a plurality of sensors positioned on the region of the limb, each sensor of the plurality of sensors configured to acquire, from the at least one vein, a respective acoustic signal of a plurality of acoustic signals in response to the pressure cuff releasing the pressure to the region;
a computing system having one or more processors in communication with the plurality of sensors, the computing system configured to:
generate, according to the plurality of acoustic signals, a corresponding plurality of metrics; and
determine, according to the plurality of metrics, a likelihood of a vascular condition in the at least one vein in the limb of the subject.

2. The system of claim 1, wherein the computing system is further configured to:
determine that the likelihood satisfies a threshold to identify the presence or absence of the vascular condition; and
provide, responsive the likelihood satisfying the threshold, an output to indicate the presence of the vascular condition of the at least one vein.

3. The system of claim 1, wherein the computing system is further configured to:
determine that the likelihood does not satisfy a threshold to identify the presence or absence of vascular condition; and
provide, responsive the likelihood not satisfying the threshold, an output to indicate the absence of the vascular condition in the at least one vein.

4. The system of claim 1, wherein the computing system is further configured to apply the plurality of metrics to a machine learning (ML) model, the ML model trained using a training dataset comprising a plurality of examples, each example of the plurality of examples identifying (i) a respective second plurality of metrics generated from a second plurality of acoustic signals acquired from a respective limb, (ii) a label indicating a presence or absence of the vascular condition in the respective limb, and (iii) data derived from ultrasound imaging to measure a compliance of at least one respective vein in the respective limb.

5. The system of claim 1, wherein the computing system is further configured to:
receive clinical data associated with the subject, the clinical data identifying at least one of: (i) demographic information, (ii) usage of pharmaceutical, or (iii) co-morbidity;
receive a clinical metric identifying one or more symptoms in the subject associated with the vascular condition; and
determine, in accordance with at least one of the clinical data or the clinical metric, the likelihood of the vascular condition in the at least one vein.

6. The system of claim 1, wherein the computing system is further configured to convert the plurality of acoustic signals from a time domain to a frequency domain to generate the plurality of metrics, each of the plurality of metrics identifying a respective coefficients from a discrete Fourier series.

7. The system of claim 1, wherein the computing system is further configured to aggregate acoustic measurements from the plurality of sensors into the plurality of acoustic signals according to a respective position of each sensor of the plurality of sensors on the region of the limb.

8. The system of claim 1, wherein the computing system is further configured to:
generate, according to a second plurality of acoustic signals acquired subsequent to administration of a treatment after acquisition of the plurality of acoustic signals, a corresponding second plurality of metrics;
determine, according to the second plurality of metrics, a second likelihood of the vascular condition in the at least one vein in the limb of the subject; and
determine, based at least on the likelihood and the second likelihood, a progress metric of the vascular condition in the subject.

9. The system of claim 1, further comprising a wearable activity tracker fittable around the region of the limb, the wearable activity tracker comprising the pressure cuff and the plurality of sensors in communication with the computing system to send an indication of a change in the vascular condition in the limb of the subject.

10. The system of claim 1, wherein the pressure cuff is secured against the region comprising at least one of a calf region or a thigh region of a leg of the subject, or an arm of the subject, the pressure cuff further configured to radially compress the region.

11. The system of claim 1, wherein the plurality of sensors further comprises a plurality of piezoelectric sensors arranged radially around the region, the plurality of piezoelectric sensors configured to acquire a plurality of acoustic signals through one or more layers of tissue, muscles, arteries, or veins in the limb of the subject.

12. The system of claim 1, wherein the vascular condition comprises at least one of: (i) deep vein thrombosis (DVT), (ii) post thrombotic syndrome (PTS), (iii) chronic venous insufficiency, (iv) peripheral vascular disease, (v) limb ischemia, (vi) phlebitis, (vii) thromboangiitis obliterans, or (viii) lymphedema.

13. A method of determining likelihood of vascular conditions using acoustic measurements of veins in limbs of subjects, comprising:
receiving, by a computing system, from a plurality of sensors positioned on a region of a limb of a subject, a plurality of acoustic signals on at least one vein in the limb, in response to a pressure cuff releasing pressure to the region;
generating, by the computing system, according to the plurality of acoustic signals, a corresponding plurality of metrics; and
determining, by the computing system, according to the plurality of metrics, a likelihood of a vascular condition in the at least one vein in the limb of the subject.

14. The method of claim 13, further comprising:
determining, by the computing system, that the likelihood satisfies a threshold to identify the presence or absence of the vascular condition; and
providing, by the computing system, responsive the likelihood satisfying the threshold, an output to indicate the presence of the vascular condition of the at least one vein.

15. The method of claim 13, further comprising:
determining, by the computing system, that the likelihood does not satisfy a threshold to identify the presence or absence of vascular condition; and
providing, by the computing system, responsive the likelihood not satisfying the threshold, an output to indicate the absence of the vascular condition in the at least one vein.

16. The method of claim 13, wherein determining the likelihood further comprises applying the plurality of metrics to a machine learning (ML) model, the ML model trained using a training dataset comprising a plurality of examples, each example of the plurality of examples identifying (i) a respective second plurality of metrics generated from a second plurality of acoustic signals acquired from a respective limb, (ii) a label indicating a presence or absence of the vascular condition in the respective limb, and (iii) data derived from ultrasound imaging to measure a compliance of at least one respective vein in the respective limb.

17. The method of claim 13, further comprising:
receiving, by the computing system, clinical data associated with the subject, the clinical data identifying at least one of: (i) demographic information, (ii) usage of pharmaceutical, or (iii) co-morbidity;
receiving, by the computing system, a clinical metric identifying one or more symptoms in the subject associated with the vascular condition; and
determining, by the computing system, in accordance with at least one of the clinical data or the clinical metric, the likelihood of the vascular condition in the at least one vein.

18. The method of claim 13, wherein generating the plurality of metrics further comprises converting the plurality of acoustic signals from a time domain to a frequency domain to generate the plurality of metrics, each of the plurality of metrics identifying a respective coefficients from a discrete Fourier series.

19. The method of claim 13, further comprising aggregating, by the computing system, acoustic measurements from the plurality of sensors into the plurality of acoustic signals according to a respective position of each sensor of the plurality of sensors on the region of the limb.

20. The method of claim 11, wherein the vascular condition comprises at least one of: (i) deep vein thrombosis (DVT), (ii) post thrombotic syndrome (PTS), (iii) chronic venous insufficiency, (iv) peripheral vascular disease, (v) limb ischemia, (vi) phlebitis, (vii) thromboangiitis obliterans, or (viii) lymphedema.
